(19) United States
(12) Patent Application Publication
(10) Pub. No.: US 2018/0182470 A1
(43) Pub. Date: Jun. 28, 2018

(54) AGGREGATED ELECTRONIC HEALTH RECORD BASED, MASSIVELY SCALABLE AND DYNAMICALLY ADJUSTABLE CLINICAL TRIAL DESIGN AND ENROLLMENT PROCEDURE

(71) Applicant: RAVI K. KALATHIL, DENVER, CO (US)

(72) Inventor: RAVI K. KALATHIL, DENVER, CO (US)

(21) Appl. No.: 15/905,628

(22) Filed: Feb. 26, 2018

Related U.S. Application Data
(63) Continuation of application No. 14/623,285, filed on Feb. 16, 2015.
Application No. 14/623,285 is a continuation-in-part of application No. 13/839,539, filed on Mar. 15, 2013, now abandoned.

(51) Int. Cl. G16H 10/20; G16H 40/20; G16H 10/60

(52) U.S. Cl. CPC G16H 10/20; G16H 10/60; G16H 40/20

(57) ABSTRACT

Adequate patient enrollment and participation in different design stages of a clinical trial is facilitated and scaled by dynamically adjusting clinical trial criteria relative to characteristics and conditions of massive numbers of patients whose medical records have been aggregated in databases in compliance with patient privacy and confidentiality laws and regulations. Patient participation results without intervention by multiple providers of healthcare services, and by directly identifying and communicating with qualified patients while maintaining patient privacy and compliance requirements as required by law.

### CROSS REFERENCE TO RELATED APPLICATION

This invention is a continuation-in-part of the invention described in U.S. application Ser. No. 13/839,539, filed Mar. 15, 2013, entitled Payment Request-Triggered, Pull-Based Collection of Electronic Health Records, invented by the inventor hereof. The subject matter described in this prior US patent application is fully incorporated herein by this reference.

### FIELD OF THE INVENTION

This invention relates principally to designing clinical trials. More particularly, the present invention relates to a new and improved procedure which obtains the medical records of a massive number of patients in compliance with patient privacy and confidentiality laws and regulations and which effectively adjusts or reformulates clinical trial criteria to identify suitable participants when designing a clinical trial.

Beneficial effects of the invention include, among other things, an increased efficiency in designing clinical trials, an enhanced probability of successfully completing clinical trials, a reduction in the amount of time and cost required to design and conduct clinical trials, and an increased capability of conducting significantly larger numbers of clinical trials for increasingly customized medical therapies.

### BACKGROUND OF THE INVENTION

Clinical trials are research studies involving humans which evaluate the safety and efficacy of medical devices and drugs that have been newly developed to treat diseases, ailments and health conditions. Clinical trials are typically conducted after the medical device or drug has been tested on animals. Clinical trials typically develop the evidence upon which governmental regulatory agencies rely when approving a medical device or drug for human use.

Clinical trials should follow strict scientific standards in order to produce reliable results. The accuracy of the clinical trial results depends on selecting a representative cohort group of individuals who are susceptible or responsive to the disease, ailment and health condition which the new medical device or drug has been developed to treat. In cases where the medical devices and drugs are intended to be effective across a broad portion of the human population, for example a measles vaccine, the cohort group selected for the clinical trial should represent a broad portion of the human population. On the other hand, a disease, ailment or health condition may afflict only a limited group of the general population, due to the specific etiological and health conditions of that limited group.

It is important to select clinical trial participants which are representative of the afflicted group. For example, the participants may be required to have specific characteristics of age, gender, ethnicity, allergies, pre-existing and other related medical conditions, and the like. In this manner, the newly developed drug or medical device is tested by a cohort group which is comparable to the same general population group to which drug or medical device is intended to be applied. Without performing the clinical trial on the relevant cohort group, the results of the clinical trial will not be reliable on the segment of the human population on which the drug or medical device is intended to be used.

Identifying suitable participants in a reliable clinical trial, and obtaining their participation in a clinical trial, are significant problems in designing a clinical trial. Information describing the medical condition of patients is protected from disclosure by patient privacy and confidentiality laws and regulations, and these laws and regulations prohibit the disclosure of most of the important and relevant information without the consent of the patient, but without access to the protected patient medical information it is difficult to locate and identify suitable participants. When the number of suitable participants is not readily determinable, it is particularly difficult to design a clinical trial that can be successfully concluded, without incurring considerable effort, expense and delay. In the past, there has been no comprehensive database of individuals and their medical conditions which can be efficiently and lawfully accessed to identify the most relevant clinical trial participants, and/or to design the clinical trial.

One previous approach to identifying participants for a clinical trial is to use a non-targeted, broadcast or public appeal approach. A particular clinical trial is promoted publicly, with the hope that a sufficient number of individuals with the desired disease, ailment or health conditions will recognize his or her applicability to the clinical trial and respond to the public solicitation. Generally speaking, such a non-targeted public appeal obtains the best response from those individuals who are already afflicted with a specific disease, ailment or health condition. The incentive for response is a potential cure or amelioration of the responder's condition. In general, the responders to such public appeals are only a very small portion of the relevant population of the relevant cohort group.

A variant of the public appeal approach involves the use of the world wide web. The US National Institute of Health (NIH) has a website, clinicaltrial.gov, that presently lists almost 140,000 clinical trials and studies. Entities which conduct clinical trials are required to register their clinical trials on this website. The clinical trials are categorized by various criteria. Interested individuals may investigate these trials on their own initiative and acquire information to enroll. Currently, the NIH website receives about 60,000 visits each day. Other websites, e.g. TrialX.com and one maintained by the University of Southern Florida, are examples of commercially available services that match potential participants with clinical trials.

In these clinical trial situations, which depend upon the responder to take the initiative to enroll, the clinical trial criteria is designated according to disease condition. Interested individuals enter their medical history, such as by downloading their medical records, and then apply them to match the published trial criteria. U.S. Pat. Nos. 7,711,580 and 7,251,609, and US patent applications 2001/0051882 and 2002/000247, are examples of procedures where interested individuals enter their demographic characteristics and medical profiles and then compare their information with clinical trial information to determine whether or not a match exists.

The success of these public appeal approaches depends on the initiative and knowledge of the prospective clinical trial participants. The value and success of the public appeal approach is limited by a prospective participant's limited understanding of the specifics of his or her medical condition, and an inability to describe those specifics as found in his or her medical record. Since the potential participants in the clinical trial voluntarily submit their medical and health information, and thereby consent to the disclosure of this otherwise private and protected information, there is no issue of compliance with patient privacy and confidentiality laws and regulations.

Another previous approach to identifying relevant participants for a clinical trial involves the organization which designs and possibly conducts the clinical trial, i.e., a “Clinical Trial Entity,” requesting physicians, hospitals and other healthcare providers to assist in identifying potential participants. The Clinical Trial Entity requests a physician or other healthcare provider to search the health records of his or her patients, looking for those patients whose medical conditions match the clinical trial criteria. On arriving at a match, the physician or healthcare provider is expected to solicit the patient to participate in the clinical trial. US patent applications 2008/0010254 and 2010/0088245 are examples of this procedure.

Using the physician or healthcare provider as an intermediary between the clinical trial entity and the potential clinical trial participant, resolves the problem of patient privacy and of accessing full medical records. However, the practical reality is that most physicians and healthcare providers are unwilling to commit the time and effort required to search individual healthcare records and actively solicit suitable patients to participate in clinical trials. The intermediated communication between suitable patients and their physicians or healthcare providers must continue until the patient agrees to the disclosure of his or her identity and medical record to the Clinical Trial Entity, which requires even further time and effort on the part of the intermediating physician or healthcare provider. The requirement for intermediation is a significant impediment in designing efficient clinical trials.

A further difficulty in intermediation between the patient and the Clinical Trial Entity is that one physician usually does not possess the entire medical health record of a particular patient. Patients frequently see different healthcare providers for different conditions and at different times of their life, so it is an unusual circumstance for one healthcare provider to possess a complete medical record of any particular individual. The lack of a complete medical record diminishes the probability of any one physician identifying suitable clinical trial participants, and thereby discourages physicians from conducting the search in the first place.

A third previous approach to the problem of identifying suitable clinical trial participants involves mining relatively large repositories of individual healthcare data, such as the records of health insurance companies, pharmacies and medical laboratories. This type of data mining attempts to match clinical trial criteria against patient medical records. In such circumstances, the patient healthcare data is annonymized to prevent disclosure of the identity of the patient. If a match is found, the physician or healthcare provider associated with that anonymized patient is requested to intermediate by soliciting his or her patient to participate in the clinical trial.

Mining insurance healthcare claims data for general healthcare trends is a well-established practice. However, the generality of this approach is not specific enough to identify relevant clinical trial participants. Insurance claims payment data typically lack the specificity and detail required to effectively evaluate whether the clinical trial criteria is matched. Data such as medical laboratory results, drug-to-drug and drug-to-food contraindications, allergies, medication lists, immunization history, family histories, physician examination notes, discharge summaries, hospitals summaries, long and short term plans of care, radiology scans, congenital conditions and genomic markers, are not typically part of insurance claims payment data, even though this information may be highly relevant or even critical to the clinical trial. The success of the healthcare claims data mining approach is also limited by the requirement for healthcare providers to intermediate communications with their patients. US patent applications 2011/0231422 and 2012/0316898 describe this mining procedure in soliciting clinical trial participants.

A fourth previous approach to identifying suitable clinical trial participants fails to address the practical and legal requirements of patient privacy. US patent applications 2012/0035954 and 2004/0034550A1, are examples of this approach. This approach uses computer-based electronic queries to directly access the medical records of the healthcare providers, attempts to match clinical trial criteria with the medical records of the patients, and thereafter directly solicits the suitable patients. The practical reality is that this process is simply not compliant with patient privacy and confidentiality laws and regulations. The medical records of patients cannot be accessed except with the consent of the patient. Direct communication with the patient other than through the patient's physician or healthcare provider is also prohibited. It is improbable that large numbers of patients would consent to having their medical records used in this manner. If a patient did consent, it is unlikely that healthcare providers would distinguish the consenting patients from the non-consenting patients in that provider's own healthcare records.

A further significant practical impediment to this fourth approach is attempting to communicate across a barrier created by the differences and complexities of the many different electronic systems which contain and manage healthcare records. A common electronic format is not used in the many different electronic medical record-keeping systems of healthcare providers, making it very difficult or impossible to extract the relevant data from the individual records and organize the extracted data in a common way for efficient usage. Even as electronic medical record keeping systems become more standardized, differences in hardware and software architectures, version levels, and network and security protocols make it inordinately complex to identify these medical records and repositories, to gain access to them and to successfully interface with them.

The above-described and other constraints have resulted in the clinical trial industry performing at a substantially sub-optimal level. According to studies of the Center for the Study of Drug Development (CSDD) at Tuft's University, 90% of all clinical trials are delayed owing to recruitment and retention issues. 15-20% of clinical trials cannot recruit a single patient, and 66% of all clinical trials do not meet enrollment (recruiting and retaining) requirements. 30% of the time spent in a trial is in recruitment, contributing to 32-40% of the costs at an average of $15,000 per enrollee. Meanwhile, a 2012 CSDD study established that from 2002 to 2012, trial criteria have increased from 31 to 50 parameters. Another 2012 study by Scannell and Warrington established that since 1950, for every 1 billion dollars spent, the number of drugs approved has halved every 9 years, resulting in a current number that is 80 times lower than the number in 1950, due to the effect of Eroom's law which is analogous to the reverse of Moore's law in computing.

The problem of identifying suitable clinical trial participants is further exacerbated considerably as drugs and healthcare therapies become more etiologically and genomically customized. In contrast to baseline therapies which have general effectiveness for broad segments of the entire population, drugs and other interventions which are customized to specific etiologies (total disease and health states), genomes, bio-markers, molecular biologies, enzyme toxicologies, etc., are focused on much smaller segments of the general population. These newly developed customized therapies must be tested in clinical trials, but the problems of identifying relevant cohort groups for customized therapy clinical trials are exacerbated by the limited access to qualified clinical trial participants who possess the specific health conditions which make them suitable participants in such clinical trials.

For example, a new therapy, even when applied to a specific medical condition (such as colorectal cancer), is often found to be effective for a percentage of the cohorts of the clinical trial group made up of individuals characterized by certain biomarkers. With relatively lower levels of effort, compared with developing the original therapy, pharmaceutical and biotech companies may adapt the original therapy to apply to individuals with different biomarkers and thereby achieve greater efficacy for portions of the cohort group. This “branching” capability represents a significant evolution in customizing medicine. However, branching is often constrained by limitations of identifying, accessing and enrolling patients with very specific etiologies as participants in clinical trials. Efficiencies in the identification, enrollment and management of clinical trials are critical in the etiological and genomic customization of medical therapies.

The problems of designing effective and relevant clinical trials are not just limited to identifying individuals who are relevant prospective participants. Contacting and communicating with the prospective participants in an effort to enroll them in the clinical trial is time-consuming, whether conducted by the healthcare provider in an intermediary capacity or whether conducted by an administrator of the Clinical Trial Entity after obtaining patient consent. Of the number of qualified prospective participants, only a limited number will respond favorably to a solicitation, and of those favorably responding individuals, an even lesser number will agree to enroll. A significant percentage of those who agree to enroll will not qualify under applicable government regulations. A percentage of those who qualify will withdraw before or after the clinical trial commences. Clinical trial entities must anticipate such attrition and reductions, in order to have a sufficient number of residual participants to complete the clinical trial and achieve meaningful results. In the past, clinical trial entities had to make guesses of whether the number of enrolled participants was sufficient. Since there was no effective method to predict the number of suitable prospective participants who will enroll, qualify and ultimately complete the clinical trial, excessive numbers of participants were enrolled as a cushion to achieve a successful completion of the clinical trial. In cases where the number of prospective participants proved to be insufficient after the clinical trial commenced, the clinical trial must be terminated prematurely, resulting in an inconclusive outcome.

Similar problems exist with respect to the costs of and time delays associated with a clinical trial. At the present time, the costs of recruiting clinical trial participants exceeds 30% of the overall cost of the trial. The difficulties in identifying relevant clinical trial participants, enrolling them, qualifying them, and maintaining their participation throughout the duration of the clinical trial, introduces unpredictable time delays and costs in bringing the medical device, drugs or therapies to market. Since clinical trials constitute a significant portion of the cost of bringing a newly developed medical therapy to market, it is very important to design a clinical trial which can be completed and which achieves reliable and sufficient results. Even more importantly, as newly developed therapies become more specific in their utilization, it is important to counterbalance decisions involving the cost of developing a new medical therapy against the market for that new therapy, to determine whether the developmental effort is justified by economic feasibility of marketing that therapy. In the past, a reliable and convenient basis to make economic feasibility evaluations of new medical therapies has been limited or nonexistent.

The ability to make reliable evaluations of the economic feasibility, and the ability to contain costs while achieving higher efficiencies in designing a clinical trial, are becoming increasingly important in view of the number of clinical trials conducted presently and to be conducted in the future. Currently worldwide, there are over fifty thousand active clinical trials involving about fifteen million participants in any year. With the expected expansion of customized therapies, it is anticipated that the number of future clinical trials, and the number of participants involved in such customized therapy clinical trials, could increase by two or more orders of magnitude. Efficiencies in the design, enrollment and management of clinical trials are increasingly becoming more critical to etiological and genomic customization of medical therapies.

### SUMMARY OF THE INVENTION

This invention involves a process for rapidly and accurately identifying suitable clinical trial participants, and thereafter providing a reliable basis for predicting the number of suitable participants who will respond to a solicitation, enroll in the clinical trial, qualify as participants and complete the clinical trial. In addition, the commercial market feasibility of a new medical therapy is determined by estimating the number of patients who would consume the new therapy. After a determination of feasibility, a fast, efficient, reliable and scalable process is established to access, solicit and enroll qualified patients in the clinical trial.

The information upon which to identify prospective participants is based on the evaluation of a full medical record of each prospective participant. The full medical records of massive numbers of prospective participants are aggregated in compliance with patient privacy and confidentiality laws and regulations, without the intermediation of healthcare providers. The access to the full medical records of massive numbers of prospective participants allows participants to be selected who have etiologies and conditions which match more specific clinical trial criteria, thereby facilitating economy and reducing cost when designing the clinical trial. Once identified, the prospective participants are efficiently solicited and enrolled.

Interacting with the full medical records of massive numbers of patients allows the clinical trial criteria to be adjusted or reformulated on a dynamic basis while designing the clinical trial. An adequate number of prospective participants is straightforwardly estimated at each stage of designing the clinical trial. Excessive or insufficient numbers of prospective participants are avoided without compromising the clinical trial, by dynamically adjusting the clinical trial criteria. Dynamically adjusting the clinical trial criteria relative to the specific etiological conditions of the prospective participants assures an adequate number of suitable participants from a massive pool of prospective participants. The identified participants constitute a statistically relevant sample size necessary to achieve a meaningful outcome from the clinical trial. Dynamically adjusting the clinical trial criteria also contains the cost and minimizes the delay of designing and conducting the clinical trial. The level of specificity which is available from dynamically adjusting the clinical trial criteria is essential when testing customized therapies that have been altered from baseline therapies, in order to evaluate efficacy for specific genomic and etiological characteristics of a limited segment of the general population.

An opportunity to wait at each stage of designing the clinical trial is also available from the present invention. The opportunity to wait increases the possibility that an adequate number of suitable patients will become available as potential participants. The pool of potential participants is constantly changing, due to new patients entering the massive pool of potential participants, due to the changing medical and health conditions of existing patients in the pool of potential participants, and due to the variable numbers of patients responding to renewed solicitations. The variations in the number and health conditions of the patients are recognized automatically and continuously over time, giving rise to the possibility that waiting will result in identifying an optimal number of suitable participants.

The costs of researching and developing new medical therapies can also be evaluated against the economic feasibility of market consumption of these new medical therapies. The costs are evaluated relative to economic feasibility thresholds determined from the medical records of the massive number of patients. Such evaluations are determined by dynamically adjusting the clinical trial criteria and thereby developing information describing the number of patients who will become probable consumers of the proposed new medical therapy.

In accordance with the invention, a method of designing a clinical trial involves aggregating patient medical records of multiple patients to establish a comprehensive database of the patient medical records of the multiple patients. The medical record of each patient in the database includes information describing the characteristics and conditions of each patient. The characteristics and conditions of a first group of patients in the database are established by collecting a basic electronic healthcare record (EHR) of a patient from a healthcare insurer or an entity responsible for payment of healthcare expenses (Payer). The Payer compensates an individual or healthcare-providing entity (Provider) which delivers healthcare products and services (Healthcare) to each patient in the first group. Patient consent is not required to collect payment data from the Payers for the first group of patients. The patient payment data is collected under business associate agreements that ensure privacy and confidentiality standards regarding the use and dissemination of the data. The collected payment data is then converted to a Basic EHR describing the Healthcare delivered by a Provider to the specific patient.

The characteristics and conditions of a second group of patients in the database are established by collecting more comprehensive EHR data directly from a Provider for each instance of the Provider delivering Healthcare to the patient, in response to the Provider submitting a payment request to the Payer, and aggregating the collected EHR data with any basic EHR data to create augmented EHR data for each patient in the second group. The patients in the second group have a relatively higher degree of specificity of characteristics and health conditions than the patients in the first group. With the database established, the clinical trial criteria is set and compared to the characteristics and health conditions of each patient in the first and second groups. Suitable patients are identified from the database who have characteristics and health conditions which match the selected clinical trial criteria, and the clinical trial is designed and conducted by reference to the identified patients.

Designing the clinical trial is facilitated by aggregating the comprehensive medical information of massive numbers of patients in compliance with existing patient privacy and confidentiality laws and regulations. The medical information is collected automatically in response to payment requests and without intermediation from Providers. Compliance with patient privacy and confidentiality laws and regulations results from designating the entity (Aggregator) which aggregates the augmented EHR data of the second group of patients as a Provider. With such a designation, the augmented EHR data of the patients in the second group is directly collected in an automated manner directly from the other Providers who render Healthcare to the patient. The patient medical record data is collected under Federal and State statutes, using Federal standards such as Meaningful Use or other interoperability protocols that allow Providers to collect medical data from other Providers as part of delivering

Healthcare to a patient.

A central entity in this invention, which functions both as an Aggregator and as a Provider, offers the advantage of dis-intermediating and scaling clinical trials. Aggregation allows a full data set (full EHRs for a very large set of patients) to be matched against clinical trial criteria, and the Provider status of the Aggregator also allows the Aggregator to have access to the medical records of patients and to solicit the patients in the event of a match.

The method of the invention involves identifying a first group of suitable patients in the database who have characteristics and conditions which match the selected clinical trial criteria, determining that the number of first identified patients is inadequate to continue designing the clinical trial, changing at least one of the characteristics or conditions of the clinical trial criteria to create adjusted or reformulated clinical trial criteria, and identifying a second group of suitable patients from the database who have characteristics and conditions which match the reformed clinical trial criteria. In such circumstances, the second identified patients differ in number from the first identified patients. The clinical trial is then designed and conducted by reference to the second group of suitable patients.

In addition, the number of patients and their medical records in the database are continuously changed or updated as the characteristics and conditions of the patients continuously change and patients continuously receive Healthcare. When the number of identified second patients is inadequate to continue designing the clinical trial, the procedure offers the opportunity to wait for the patient medical records to update. Thereafter, matching the trial criteria with the updated database permits the identification of a different member of suitable patients who have characteristics and health conditions which match the clinical trial criteria. The possibility of changing the number of suitable patients identified after the patient records have updated, may facilitate designing the clinical trial.

These features of the invention permit determinations of whether an adequate number of suitable patients are identified at the solicitation, participation, enrollment and initiation stages of the clinical trial design procedure. The economic or market feasibility of developing a new medical therapy is also determined by use of the clinical trial criteria and the relative proportions of patients in the different groups of patients in the database.

The health condition or etiology of the patient can be further augmented beyond the Basic and Augmented EHR databases by including genomic and post-genomic characteristics to the database. These additional characteristics are utilized similarly to match clinical trial criteria and determine the feasibility for a clinical trial.

Other aspects and features of the invention, as well as a more complete understanding of the present invention and its scope may be obtained from the accompanying drawings, which are briefly summarized below, from the following description of presently a preferred embodiments of the invention, and from the appended claims.

### BRIEF DESCRIPTION OF THE DRAWINGS

FIGS. 1A, 1B, 1C and 1D collectively constitute a flow chart of actions involved in a method or procedure of designing a clinical trial, in which the present invention is embodied.

FIG. 2 is a block diagram of exemplary data processing and communication computer systems maintained by a Clinical Trial Entity and an Aggregator, and a public communication network, all of which are used in facilitating execution of certain aspects of the procedure shown in FIGS. 1A-1D.

FIG. 3 is a block diagram showing entities, actions and communications involved by an Aggregator in aggregating and establishing a comprehensive database of patient medical records for a massive number of patients, in accordance with a part of the procedure shown in FIGS. 1A-1D.

### DETAILED DESCRIPTION

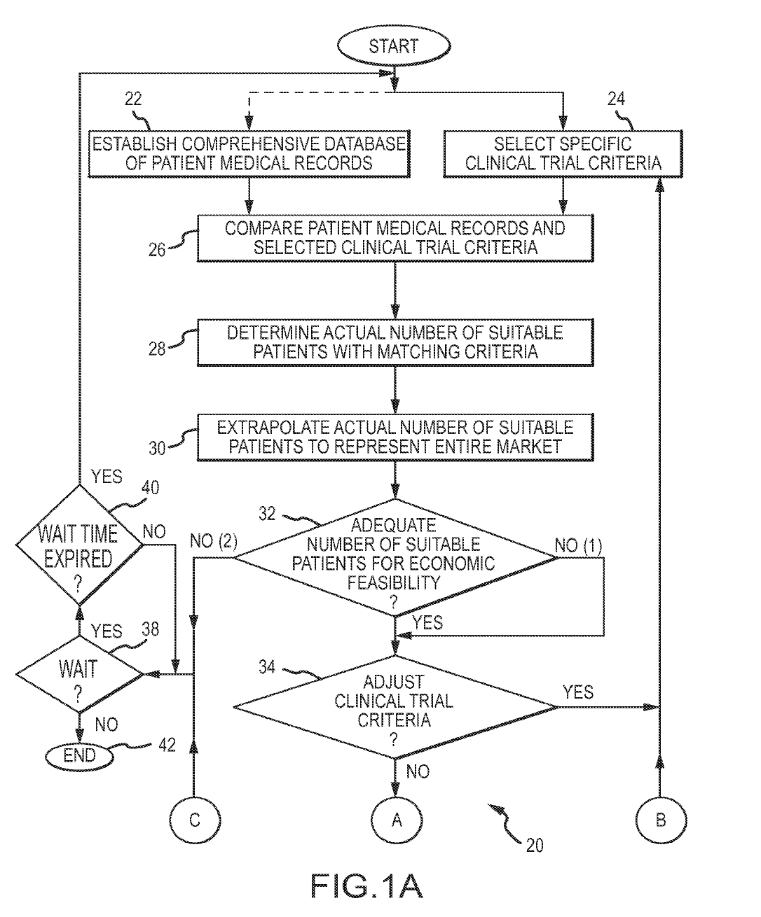

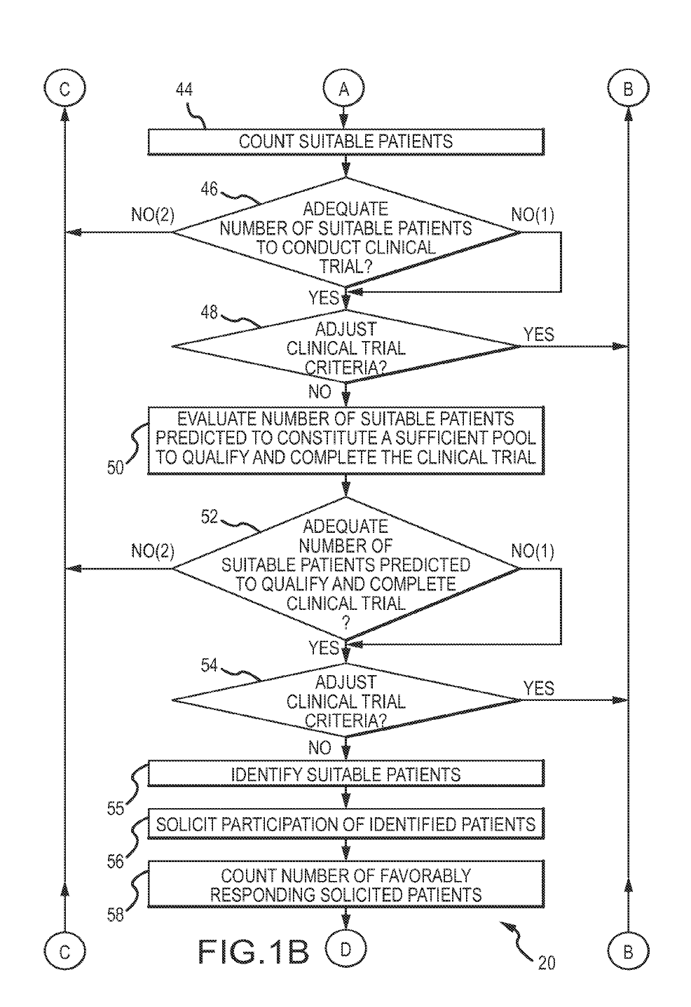

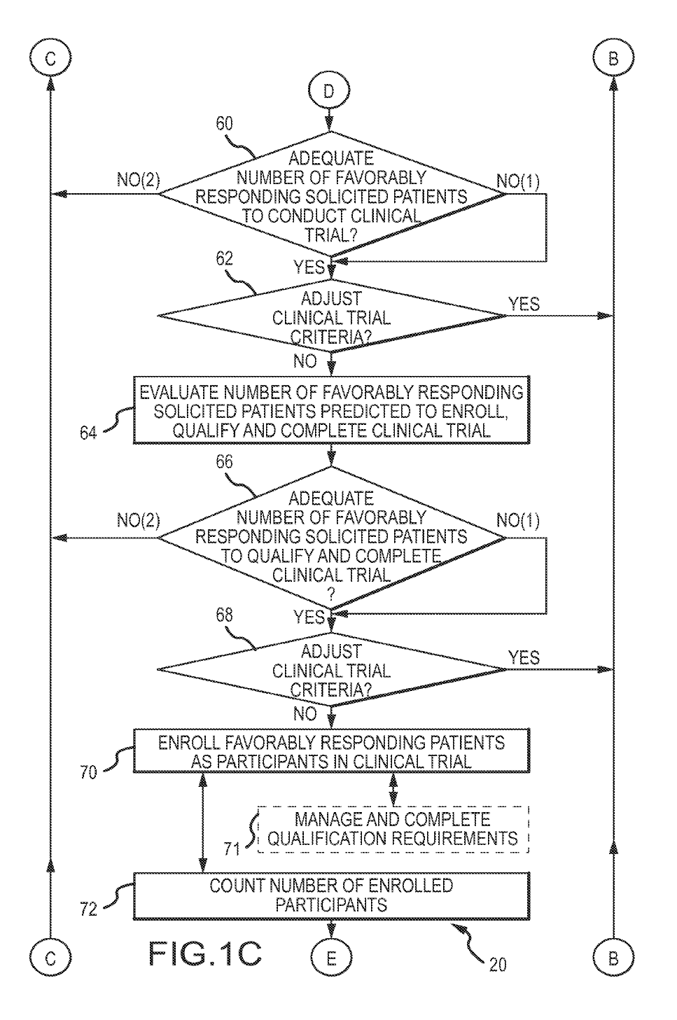

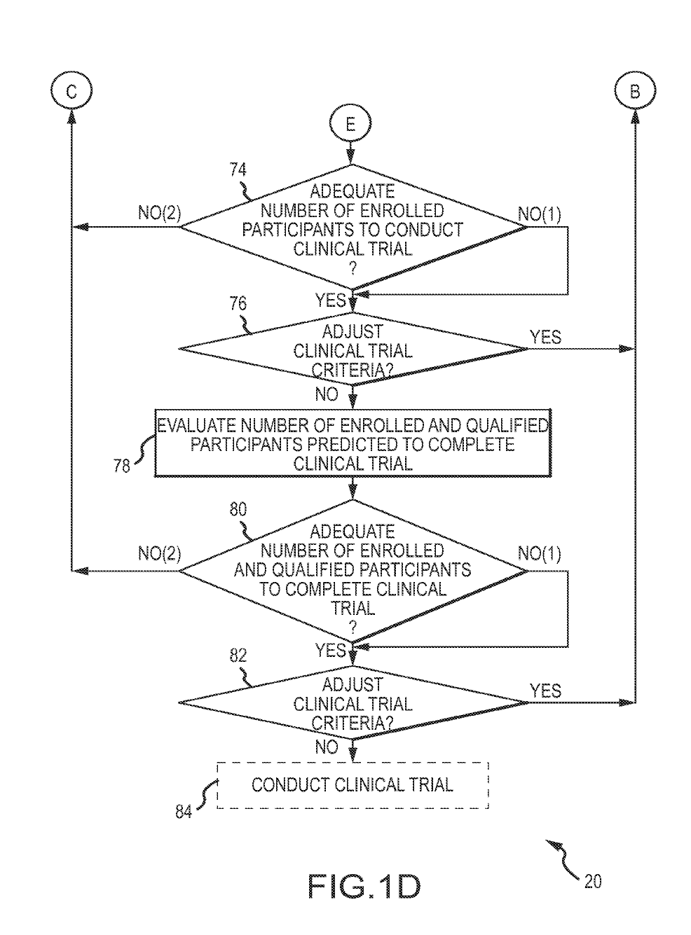

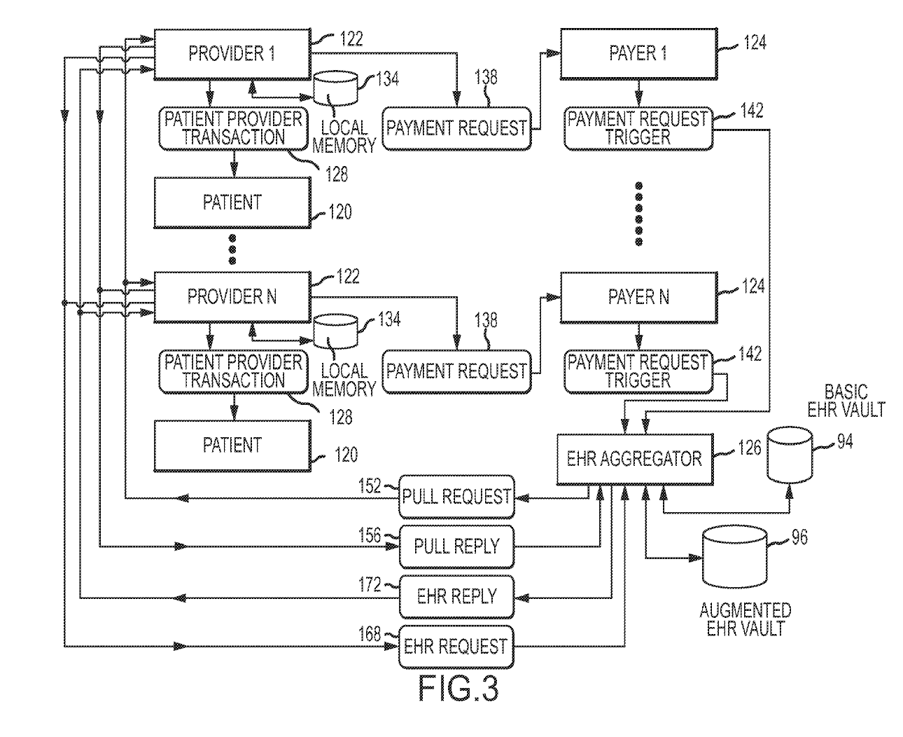

The present invention is embodied in a method or procedure 20 of designing clinical trials, shown in FIGS. 1A-1D. In general, the procedure 20 involves establishing a comprehensive database of patient medical records at 22 for a massive number of patients, for example millions of patients, by aggregating the medical records in a database. The degree of detail or specificity of the medical record of each patient varies, so the database established at 22 for some patients extends only to a basic health record, while other patients have a more extensive medical record that also includes details derived from the records of the Healthcare (medical products and services) delivered by Providers (individuals and entities that supply Healthcare), while still other patients have an even more comprehensive medical record that also includes such additional information as genomic sequences and markers and other more detailed descriptions of specific health and medical characteristics. Establishing the comprehensive database of patient medical records at 22 involves aggregating and using the patient medical records in compliance with patient privacy and confidentiality laws and regulations. One technique of aggregating the medical records of massive numbers of patients is described generally below in connection with FIG. 3, and more specifically in the above referenced prior U.S. patent application Ser. No. 13/839,539.

The specific health and medical conditions desired in the participants in the clinical trial are selected as clinical trial criteria at 24. Trial criteria are selected from a predefined table of criteria. The criteria correspond to specific patient characteristics, patient conditions and health and medical information which will typically be contained in the medical records of patients. The trial criteria include data such as age, gender, address, race, previous diseases, previous medical procedures, drug histories, allergies, congenital conditions, epidemiologies, and genome characteristics, and the like, among other things. These criteria are referred to herein as etiologies. The trial criteria/etiologies are reflected in the database established at 22.

A committee of medical and regulatory experts establishes the characteristics which define the entries in the predefined trial criteria table. The committee of experts also approves any changes to the criteria table. In this manner, the committee of experts assures that there is a singular medically-specific definition and designation for each particular criteria/etiologies. The use of specific nomenclature assures that each clinical trial criteria may be designated and identified only in a singular manner, thereby avoiding confusion among the various criteria/etiologies. The committee of medical and regulatory experts which define the entries in the trial criteria table are employed by or associated with the entity which aggregates the patient medical records of multiple patients in the database, referred to herein as an “Aggregator.” The function of the committee of medical and regulatory experts employed by the Aggregator is to assure that the criteria/etiologies of the patients in the database are specifically designated and free of substantial confusion with and distinguishable from other criteria/etiologies.

An Administrator of the entity which designs and possibly conducts the clinical trial, herein referred to as a “Clinical Trial Entity,” selects a combination of multiple entries from the pre-defined criteria table to establish the initial trial criteria at 24 which is used in designing the clinical trial. The Administrator of the Clinical Trial Entity may be aided in the selection at 24 by other medical experts employed by the Clinical Trial Entity. The initial trial criteria is selected by the Administrator at 24 with the view toward identifying those potential participants in the clinical trial which will provide the most reliable information for evaluating the efficacy of the newly developed drug or therapy which is the subject of the clinical trial. Changes to the selected clinical trial criteria may occur as a result of the dynamic adjustment features of the procedure 20, which are described below, as overseen by the Administrator and possibly by medical experts employed by the Clinical Trial Entity.

The clinical trial criteria selected at 24 are thereafter compared by the Aggregator at 26 with the medical records of the patients in the database established at 22. The comparison is facilitated as a result of the Aggregator organizing the specific criteria/patient etiologies in the database established at 22 so that the etiologies may be searched and matched efficiently. At 28, a number of suitable patients with characteristics and health conditions matching the clinical trial criteria selected at 24 is determined. The number of suitable patients with matching criteria determined at 28 is thereafter used in the procedure 20 as the basis to design and organize the clinical trial.

In order to maintain patient privacy, the Administrator of the Clinical Trial Entity interacts with a Liaison from the Aggregator in order to accomplish the comparison at 26, thereby preventing access by the Clinical Trial Entity to the database of information created by the Aggregator. In general, the Administrator of the Clinical Trial Entity supplies the list of clinical trial criteria to the Liaison of the Aggregator. The Liaison oversees the comparison executed by the Aggregator and supplies the number of suitable patients with matching etiologies to the Administrator. The names of the patients in the details of their medical records are not disclosed at this stage of the procedure 20. Consequently, the private medical information of the patients is maintained confidential by the Aggregator, and is not disclosed at this stage of the procedure 20. At this stage of the procedure, the Administrator and the Clinical Trial Entity are principally interested in the numbers of patients having health and medical conditions which match the clinical trial criteria.

The function of the Liaison is predominantly automated to serve as an interface between the Clinical Trial Entity and the Aggregator. The Administrator's functions are facilitated by computer systems and user interfaces, provided either by the Clinical Trial Entity or the Aggregator, to automate as many of the tasks performed by the Administrator as are feasible. Manual or human tasks that need to be performed, particularly those requiring interfacing between the Clinical Trial Entity and the Aggregator, are generally handled by either the Administrator or the Liaison. Given the number of trials managed concurrently by the Aggregator, most of the activities of the Liaison and the Administrator will be automated.

As is discussed in more detail below, certain stages of the clinical trial design procedure 20 involve dynamically adjusting the clinical trial criteria selected at 24. Among other things, dynamic adjustment of the clinical trial criteria facilitates a determination of the economic feasibility of developing the new medical therapy and facilitates efficiently and effectively designing and completing the clinical trial. In general, the dynamic adjustment aspects of the procedure 20 involve iteratively changing the selected clinical trial criteria at 24 to evaluate and optimize the number of suitable participants determined at 28, in a way which ensures an efficient and effective design of the clinical trial.

The dynamic adjustment capability of the procedure 20 also facilitates a determination of the economic feasibility of researching, developing and marketing the new medical therapy. In general, economic feasibility is accomplished by the Clinical Trial Entity at 30, by extrapolating the number of suitable patients determined at 28 to obtain a reasonable expectation of the total number of patients in the entire population which possess the etiological characteristics which will be served by the newly developed medical therapy. As such, the number of patients extrapolated at 30 constitute a reasonable approximation of the economic market for consuming the newly developed therapy.

The extrapolation performed at 30 is based on the numbers of patients and their medical records in the database established at 22. After the extrapolation at 30, a determination is made at 32 as to whether a sufficient market of consuming patients exists to justify the costs of researching and developing the new medical therapy. If there is insufficient market feasibility, as determined by a no (1) negative determination at 32, an affirmative determination at 34 results in the process flow moving to 24, where the clinical trial criteria is adjusted by changing the degree of specificity of the clinical trial criteria. Then, using the adjusted clinical trial criteria, the actions identified at 26, 28, 30 and 32 are performed again to evaluate market feasibility.

The adjustment which results from the no (1) negative determination at 32 will increase the number of qualified patients with matching criteria, determined at 28, when the level of specificity of the clinical trial criteria at 24 is decreased by eliminating one or more of the patient etiologies previously selected from the clinical trial criteria. On the other hand, the adjustment can also decrease the number of patients with matching criteria by increasing the level of specificity of the clinical trial criteria. Dynamically adjusting the clinical trial criteria in this manner to increase or decrease the level of specificity of etiologies permits exploring the limits of the economically feasible market for the new medical therapy.

Another important feature of the procedure 20 relates to the type of action which may be taken in response to a circumstance where adjusting the clinical trial criteria does not result in meeting a desired threshold. When determining economic feasibility, an inability to achieve sufficient economic feasibility after adjustment of the clinical trial criteria is represented by a no (2) negative determination at 32. In that case, the procedure 20 offers an opportunity to wait at 38 until for a desired amount of time determined at 40. A decision to wait at 38 for a desired amount of time at 40 allows more patient medical records to be accumulated in the database established at 22, and allows the medical records of patients previously in the database to change due to changes in the health and medical conditions of patients occurring over time. The opportunity to wait at 38 and 40 is a viable option because the database established at 22 is updated on a continuous basis by the Aggregator with the addition of medical records of new patients and changes to the health and medical conditions of existing patients.

Waiting the desired amount of time, as determined at 38 and 40, offers the possibility that the updated information in the database established at 22 will contain adequate information to overcome the threshold circumstance which the previous dynamic adjustment of the clinical trial criteria could not overcome. If the decision is to wait at 38, the procedure 20 is again executed at the expiration of the time established at 40. Executing the procedure 20 after the wait time determines whether an adequate number of new patients and patients with changed medical records are now present in the database established at 22 to evaluate economic feasibility, or, as discussed below, to change the number of participants in the clinical trial. Of course, if the decision at 38 is not to wait, the procedure 20 ends at 42. The Administrator can also, at any point, interrupt the wait time period and thereby cause its expiration. The process flows, in this case, would follow the same routes as though the wait time had expired without interruption.

The opportunity for the Clinical Trial Entity to dynamically adjust the clinical trial criteria to meet significant thresholds at each step of designing the clinical trial significantly improves the typical procedure involved in designing and conducting a successful clinical trial. The information available from dynamic adjustment optimizes the design of each stage of the clinical trial. In general and in reference to FIGS. 1B-1D, dynamic adjustment is used to increase or decrease the number of suitable identified patients at 44, 46 and 48; to evaluate whether the number of identified patients constitutes an adequate pool of participants to complete the clinical trial at 50, 52 and 54; to increase or decrease the number of patients who respond to a direct solicitation for participation in the clinical trial at 56, 58, 60 and 62; to evaluate whether the number of favorably responding patients constitutes an adequate pool of favorably responding patients to qualify and complete the clinical trial at 64, 66 and 68; to increase or decrease the number of favorably responding patients who actually enroll as qualified participants in the clinical trial at 70, 72, 74 and 76; and to evaluate whether the number of enrolled patients constitutes an adequate pool of participants to complete the clinical trial at 78, 80 and 82. At each of these procedural stages of designing the clinical trial, there is an opportunity to wait at 38 for the expiration of a predetermined time at 40. Waiting might facilitate meeting the desired thresholds which were otherwise not possible to meet prior to the expiration of the waiting time, or to end the procedure 20 at 42. Satisfying all of these conditions or thresholds when designing the clinical trial facilitates successfully conducting the clinical trial at 84.

The capability to adjust the scope of the clinical trial by adjusting the trial criteria to achieve an adequate number of participants for economic feasibility and to facilitate the successful completion of the clinical trial, at each of the many stages of designing the clinical trial, is a significant improvement over prior clinical trial design techniques. Known prior techniques do not provide a convenient opportunity to adjust, in an informed manner, the scope of the clinical trial on a dynamic basis at each of the principal stages of designing the clinical trial. As a consequence, prior clinical trials are subject to more uncertainty with respect to cost, efficiency and successful conclusion.

In contrast, dynamically adjusting the clinical trial criteria and evaluating the results of such adjustments relative to thresholds at each stage of the clinical trial procedure 20, allows the clinical trial design to go forward with optimal efficiency, thereby avoiding excessive costs and unexpected time delays, while still ensuring that the results will provide enough reliable information to determine efficacy of the newly developed therapy. Reducing the cost of the clinical trial, and increasing the efficiency with which the clinical trial is conducted, are important factors in developing a new medical therapy, because about 30% of the cost of developing a new medical therapy is presently consumed by conducting the necessary clinical trials. Reducing the costs of the clinical trial without compromising the reliability of the results is a significant improvement over past methods of designing clinical trials.

In the past, clinical trials were not designed through the use of a comprehensive database of patient medical records that was established in compliance with patient privacy and confidentiality laws and regulations. Past clinical trials had no known capability to iteratively adjust the scope of the clinical trial by changing the clinical trial criteria using a comprehensive database of patient medical records. As a consequence, prior clinical trials typically enrolled an excessive or insufficient number of participants. Enrolling an excessive number of participants increased the cost of the clinical trial without achieving a comparable increase in information by which efficacy could be determined. Enrolling an insufficient number of participants led to a premature termination of the clinical trial due to the natural tendency of some participants to drop out before the clinical trial was completed, or led to the clinical trial delivering an insufficient amount of information by which to determine efficacy on a reliable basis.

The comprehensive database of patient medical records established by the Aggregator at 22 makes possible the dynamic adjustment of the scope of the clinical trial. Without a comprehensive database of medical patient records, there is no efficient capability to compare specifically selected clinical trial criteria with the etiologies of massive numbers of patients. Accordingly, the benefits of the present invention will not be fully realized without the ability to aggregate and establish a comprehensive database of medical records of many patients.

The aggregation of the patient EHRs also allows the Aggregator and the Clinical Trial Entity (once patient consent has been obtained), to directly push clinical trial information and solicitation to qualified patients in a very specifically targeted manner while maintaining the confidentiality of the patient. This approach is a significant improvement over current techniques of pulling in patients based on a broad notice of a clinical trial with the expectation that the patient will, on his or her own initiative or through an intermediated solicitation by a Provider, find his or her way to a clinical trial.

The comprehensive database of patient medical records, and the ability to dynamically adjust the clinical trial criteria relative to the patient health and medical conditions recorded in that comprehensive database, coupled with the capability to wait for changes in the patient's medical records or in the number of patients, and the ability to directly target and solicit qualified patients, are significant improvements in designing clinical trials and in overcoming the prior detrimental aspects of designing clinical trials, as described in greater detail below.

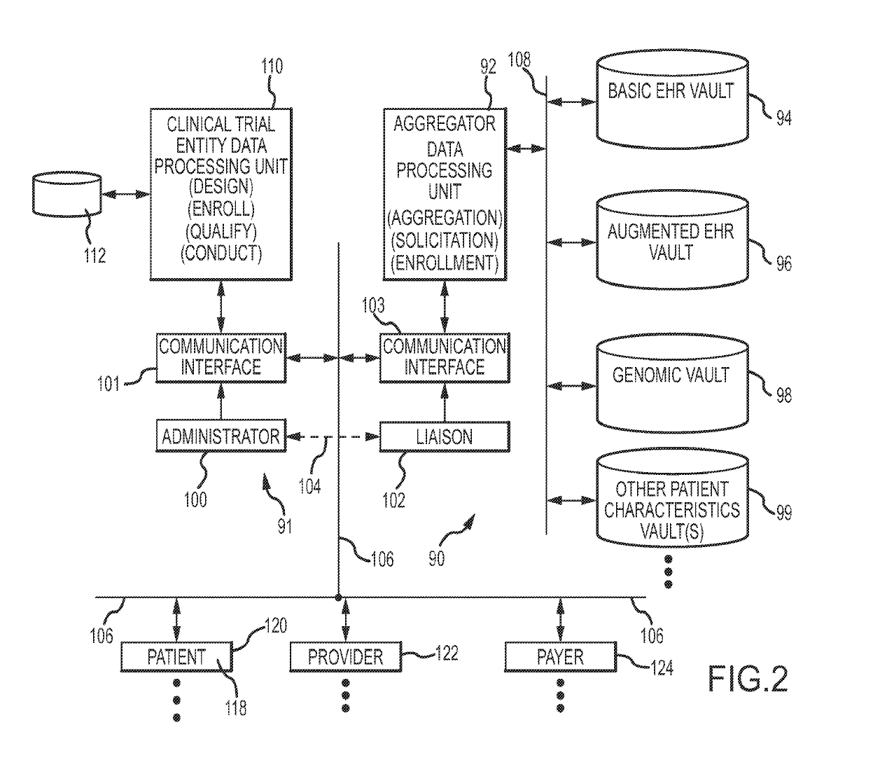

The procedure 20 is preferably executed with the aid of two separate data processing and communication computer systems 90 and 91, shown in FIG. 2. The computer system 90 is maintained and controlled by the Aggregator to establish and update the comprehensive database of patient medical records 22 (FIG. 1A). The other computer system 91 is controlled by the Clinical Trial Entity to design the clinical trial. The separate computer systems 90 and 91 prevent the Clinical Trial Entity from accessing the protected medical records of the patients aggregated by the Aggregator. Similarly, the Aggregator is prevented from accessing the protected aspects of the clinical trial procedure 20.

The Aggregator computer system 90 has a capability to solicit patients (56, FIG. 1B) to participate in the clinical trial designed by the Clinical Trial Entity, until such time as the patient agrees to participate in the clinical trial and gives consent to the Aggregator to disclose his or her identity and/or medical records to the Clinical Trial

Entity. Once the consent is given, the Clinical Trial Entity computer system 91 has the capability to directly communicate and start the enrollment process (70, FIG. 1C) with those patients who have given their consent. Specifically and only for these patients, the Clinical Trial Entity may, if required, receive copies of the medical records from the Aggregator computer system 90. The Clinical Trial Entity computer system 91 will typically be used to enroll consenting patients in the clinical trial (70 and 71, FIG. 1C), but the Clinical Trial Entity may also request the Aggregator to assist with patient enrollment, in which case Aggregator computer system 90 may also have a capability for enrollment.

The computer systems 90 and 91 automatically execute those aspects of the procedure 20 which do not require human intervention. The Administrator 100 interacts with the computer system 91 at human decision points in the procedure 20 through a communication interface 101, while executing the Clinical Trial Entity aspects of the procedure 20. The Liaison 102 interacts with the computer system 90 at human decision points in the procedure 20 through a communication interface 103, while executing the Aggregator aspects of the procedure 20. The Administrator 100 and the Liaison 102 may establish a direct communication link 104 with each other, and/or the Administrator 100 and the Liaison 102 may also communicate over a public communication network, such as the internet 106, through their respective communication interfaces 101 and 103. As described earlier, to facilitate the scale of clinical trials conducted, the functions of the Administrator 100 and the Liaison 102 are preferably automated to the maximum extent possible.

The Administrator 100 communicates with the Liaison 102 to request the Aggregator to execute the instructions of the Clinical Trial Entity when designing the procedure 20, under circumstances where access to and interaction with the patient medical records must be kept confidential, such as when suitable identified patients are solicited to participate in the clinical trial. The Liaison 102 electronically communicates with the patients over the internet 106 when issuing solicitations to participate in the clinical trial, when the solicited patient has a communication capability through the internet 106. In those circumstances when the solicited patient does not have an internet communication capability, the Liaison 102 issues communications and solicitations by an alternative communication procedure, such as regular postal service. The Administrator 100 electronically communicates over the internet 106 to transmit information and instructions to those patients who have consented to allow communication of their identity and/or medical records with the Clinical Trial Entity, and to enroll those suitable consenting patients as participants in the clinical trial.

The Administrator 100 and the Clinical Trial Entity also use the internet 106 as much as possible when qualifying enrolled patients and in conducting the clinical trial. An effective communication capability between the Administrator 100 and the Liaison 102 coordinates the functionality of the computer systems 90 and 91 when performing the clinical trial procedure 20. It is advantageous from an efficiency standpoint for as many patients 120 as possible to be connected for communication through the internet 106 to facilitate efficiency in designing the clinical trial. Efficiency is facilitated by direct communications over the internet 106 to solicit suitable patients to participate in and enroll in the clinical trial, to enroll in the clinical trial, to qualify for the clinical trial and in some cases to report results from participating in the clinical trial.

Patients 120 may also communicate over the internet 106 with either of the computer systems 90 and 91, respectively, under appropriate safeguards where those communications do not violate the privacy of medical records or adversely influence the procedure 20 performed by the Clinical Trial Entity and the Aggregator. The patients 120 may also communicate with the Administrator 100 and/or the Liaison 102 under appropriate circumstances to prevent the disclosure of confidential patient information. Patient communications from the internet 106 are managed so as to not interfere with aspects of the functionality controlled by the Aggregator and of the Clinical Trial Entity.

The Aggregator computer system 90 includes one or more data processing units 92, each of which is connected to banks of separate memories 94, 96, 98 and 99 by a system bus 108. Each of the memories 94, 96, 98 and 99 is used to store the data describing the medical records of the patients. The memories 94, 96, 98 and 99 constitute the comprehensive database of patient medical records established at 22 (FIG. 1A). All of the patients which make up the comprehensive database (22, FIG. 1A) are identified in one or more of the memories 94, 96, 98 and 99. A common unique patient identification identifies the patients in the memories. Although memories 94, 96, 98 and 99 are shown as separate, they may be combined into separately identifiable portions of a single large memory.

The memory 94 constitutes a basic electronic health record (EHR) vault, the memory 96 constitutes an augmented EHR vault, the memory 98 constitutes a genomic vault and the memory 99 collectively refers to other patient characteristics that may be collected, such as the epi genome. The data describing the medical record of each patient in each vault 94, 96, 98 and 99, varies according to the level of specificity or detail describing the patient's characteristics and health conditions, i.e. the patient's etiology, and according to the level of participation by the patient.

The EHR vault 94 stores the EHR for each patient identified in that vault 94. The patient and his or her EHR identified in the EHR vault 94 represent the lowest level of patient participation in the comprehensive database. The basic EHR includes the patient name, gender, address, date of birth, the date of a medical procedure, a disease code for the condition treated and a code for the procedure performed. Pre-existing standards define the disease and procedure codes. The information for the EHR vault 94 is derived from healthcare claims sent by a Provider to a Payer to obtain payment for Healthcare rendered to the patient. Those individuals and entities which deliver healthcare products and services to a patient are referred to herein as Providers. Providers include doctors, doctor offices, clinics, surgical centers, laboratories, hospitals, urgent care centers, pharmacies, rehabilitation centers and physical therapists. Healthcare constitutes both services and products delivered to a patient by a Provider. A Payer is a healthcare insurer or an entity responsible for paying the Provider for rendering Healthcare to a patient.

The data in the basic EHR vault 94 is collected from claims repositories maintained by Payers. Since the information in the basic EHR vault is an aggregation of claims records from various Payers, no consent of the patient is required to collect this claims data; the collection and use of the selected healthcare claims data is part of the business relationship between the Aggregator and the Payer. Claims data is regularly used to develop information describing current health trends. Collection and use of this claims data under accepted guidelines and business associate agreements is not a violation of patient privacy and confidentiality laws and regulations.

The augmented EHR vault 96 stores an augmented EHR for each patient identified in that vault 96. The information contained in the augmented EHR vault 96 includes all of the basic EHR information plus the additional information obtained from the records of a Provider who delivered Healthcare to a patient. This additional information includes data such as lab results, drug to drug allergies, food to drug allergies, food to food allergies, general allergies, discharge summaries, immunizations, untreated disease codes, family histories, and congenital conditions (e.g., Gilbert's Syndrome). This additional information is derived directly from the records of each Provider who renders Healthcare services to that patient, as is discussed below in detail in conjunction with FIG. 3. Patient consent is required to populate the augmented EHR vault 96. In addition, information contained in the augmented EHR vault 96 also includes health monitoring data supplied by the patient. Health monitoring data is currently available from consumer apps running on smartphones and other home and close-to-patient based diagnostics programs.

The information contained in the augmented EHR vault 96 is obtained from a local clinical computer system of the Provider which renders Healthcare to the particular patient, in response to an electronic request communicated to that Provider. In response, the clinical computer system (FIG. 3) of the Provider communicates the patient's healthcare information which is then recorded in the augmented EHR vault 96. The communication of the healthcare information in this manner may comply with the Meaningful Use (MU) standard required by US law or with other interoperability standards or arrangements.

A genomic vault 98 stores even more comprehensive information describing the characteristics and medical and health conditions of certain patients. The genomic vault 98 contains some or all of the sequenced genome for the patient, as well as markers for some specific genomic conditions for which tests are currently available, such as the Alzheimer marker, APOE e2/e4, the breast cancer marker, BRCA 1 & 2, and the like. Genomic information is typically beyond the healthcare record information contained in the augmented EHR vault 96 for each patient. However, if such genomic information is procured for a patient, it is stored in the genomic vault 98. In most cases, the patient will undergo the necessary tests and evaluations to derive and thereafter supply the genomic information for inclusion in the genomic vault 98. The consent of the patient is required to populate the genomic vault 98 with the patient's genomic information, unless that information is available from the health care records of the patient's Provider.

Other vaults 99 are intended to anticipate even more specific and individualized information associated with each patient, such as the epi Genome. A number of vaults 99 are provided, and each of them may be limited to a specific and individualized health or medical condition or characteristic of a patient. In general, the consent of the patient is required to populate the vaults 99 with that patient's information.

Payers encourage patients to consent to delivering their medical records to the augmented EHR vault 96. Patients who give this consent are more likely to consent to having their genome mapped and included along with their augmented EHR in the genomic record. Additionally, these patients are more likely to actively monitor their health by periodically collecting health data and communicating that data to enhance their medical records. Due to an elevated interest in health, these patients are usually receptive to monitoring, receiving, evaluating and responding to solicitations for enrollment as participants in clinical trials. Information which is not collected as described in FIG. 3 but which is supplied by patients, is entered in the vaults 94, 96, 98 and 99 by the Aggregator through the communication interface 103.

The augmented EHR vault 96 has more specific etiological information than the basic EHR vault 94, but fewer numbers of patients are identified in the augmented EHR vault 96 than the greater number of patients identified in the basic EHR vault 94. The genomic vault 98 has even more specific etiological information for each of the patients identified in that vault than the etiological information for each of the patients identified in the augmented EHR vault 96, but the number of patients identified in the genomic vault 98 is typically lesser than the number of patients identified in the augmented EHR vault 96. The vaults 99 which store even more specific etiological information of certain patients, typically identify an even fewer number of patients than the number of patients identified in the genomic vault 98.

The differing number of patients in each of the vaults 94, 96, 98 and 99, and the differing content of the etiological information describing each patient, is used in the procedure 20 (FIGS. 1A-1D) to extrapolate and predict certain information which was not previously established or used but which is important to efficiently and effectively design a clinical trial.

The Clinical Trial Entity computer system 91 includes one or more data processing units 110, each of which is connected to a memory 112 which contains the code which defines the programming instructions necessary to perform the aspects of the present invention performed by the Clinical Trial Entity, as discussed below. The communication interface 101 is connected to control each data processing unit 110.

The details of obtaining the EHR information to populate the augmented EHR vault 96, as well as establishing the comprehensive database of medical records (22, FIG. 1A) in compliance with patient privacy and confidentiality laws and regulations, are generally described in conjunction with FIG. 3, and are described more specifically in the above referenced U.S. patent application Ser. No. 13/839,539. Except in those instances where the actions and communications must be performed by humans, the actions and communications described in FIG. 3 are anticipated to be performed by electronic computer and communications devices which have been programmed to execute the functions described.

The EHRs of the patients 120 are aggregated and augmented automatically by the interaction and communication between Providers 122, Payers 124 and at least one EHR Aggregator 126. The functionality of the Aggregator 126 may be executed by the data processing units 92 of the computer system 90 (FIG. 2), or may be executed by a separate computer system which delivers the data to the vaults 94 and 96 of the computer system 90 (FIG. 2). The Aggregator 126 must attain the status of a Provider, in order to automatically or otherwise access the health records maintained by a Provider while complying with patient privacy and confidentiality laws and regulations. Patients 120 have an incentive to designate the Aggregator 126 as a Provider, because the Aggregator 126 will maintain a complete and accurate medical record of the patient. A complete and accurate medical record will facilitate the patient receiving appropriate Healthcare. The fact that a new or different Provider may access the augmented EHR vault 96 to provide Healthcare is a further incentive to the patient to designate the Aggregator 126 as a Provider. The timely comprehensiveness of the EHR data that is made available from Providers enhances the quality of Healthcare, and safety of the patient, and serves as an improved basis to manage healthcare costs.

The patients 120, Providers 122, Payers 124 and the Aggregator 126 interact with one another by communicating and taking those actions shown and explained in connection with FIG. 3. For convenience of illustration and description, FIG. 3 illustrates instances of a single patient 120 interacting with a single Provider 122 and that single Provider 122 interacting with a single Payer 124. In actual practice, a single patient 120 could interact with multiple Providers 122, and each Provider 122 could interact with multiple Payers 124.

The patient 120 begins by seeking Healthcare from a Provider 122. This relationship is established in a patient-Provider transaction 128. The patient-Provider transaction 128 involves the Provider 122 authenticating the identity of the patient seeking the Healthcare, and assures that the EHR will be established for the correctly identified patient.

As part of the patient-Provider transaction 128, the Provider 122 delivers Healthcare to the patient 120. In conjunction with delivering the Healthcare, the Provider 122 establishes the EHR that describes the Healthcare delivered to the patient. The EHR created by the Provider 122 is established in Meaningful Use (MU)-compliant form, and that MU-compliant EHR is then stored locally in a local memory 134 of the clinical computer system of the Provider 122. Providers are required by law to commence using MU digital healthcare standards and specifications which establish a format and definition of an EHR. The MU standards also establish a uniform protocol for communication and information exchange of EHRs between Providers. The principal purpose of the MU standards is to establish a basis for Providers to exchange EHR data about patients in a timely manner, thereby offering the possibilities of increased coordination and quality of Healthcare and safety of the patient, and managed healthcare costs. Although in MU is described herein as the prevailing standard for EHR storage and dissemination, other EHR and interoperability standards and arrangements could be used in accordance with the invention.

The Provider 122 thereafter seeks payment for the Healthcare delivered to the patient 120, by submitting a payment request 138 to the Payer 124 that is responsible for paying for the Healthcare delivered to the patient 120. The payment request 138 submitted by the Provider 122 to the Payer 124 is in a standardized format established by the Payer for payment requests. The standardized format for payment requests includes information which identifies the patient and the Provider, and information which contains a basic description of the Healthcare delivered by the Provider to the patient. This standardized format is required by the Payer 124 to evaluate the legitimacy and the extent of the payment request. Although not shown, in response to a proper payment request 138, the Payer 124 will send payment to the Provider 122.

The Payer 124 then transmits a payment request trigger 142 to the Aggregator 126. The payment request trigger 142 includes the identifications of the patient and the Provider and a basic description of Healthcare delivered, derived from or based on the information contained in the payment request 138. The Aggregator 126 interprets the payment request trigger as an indication that Healthcare has been delivered to the identified patient by the identified Provider. In response to the payment request trigger 142, the Aggregator 126 commences action to establish, collect and augment an EHR record for the identified patient, by collecting information from the EHR stored in the local memory 134 of the identified Provider 122.

In preparation for establishing, collecting and augmenting the EHR record for each identified patient, the Aggregator 126 extracts from the payment request trigger 142, the identity of the patient, the identity of the Provider, and the basic EHR data contained in the payment request trigger 142. The information extracted from the payment request trigger 142 is then stored by the Aggregator 126 in the basic EHR memory vault 94 (also shown in FIG. 2).

Using the extracted identification of the Provider 122 and the patient 120, the Aggregator 126 sends a pull request 152 to the identified Provider 122. The pull request 152 includes the identification of the patient 120, and constitutes a request for the Provider 122 to obtain from the local memory 134, the MU-compliant EHR data of the identified patient and to transmit that EHR back to the Aggregator 126. In addition to the identity of the patient, the pull request 152 may also include at least one aspect of the basic EHR data contained in the payment request trigger.

The Provider 122 responds to the pull request 152 in a pull reply 156. The pull reply 156 involves obtaining the MU-compliant EHR data of the identified patient from the local memory 134 and transmitting that EHR back to the Aggregator 126. The

EHR data transmitted by the Provider constitutes the major part of the pull reply 156 and includes comprehensive information describing the Healthcare delivered to the identified patient. The EHR data returned in the pull reply is more complete, compared to the basic description contained in the payment request trigger 142. Accordingly, the EHR data provided to the Aggregator 126 in the pull reply 156 is a more complete record of the Healthcare delivered to the identified patient. A complete record of the Healthcare delivered to the identified patient by the Provider is permitted under the law because the Aggregator 126 has been designated by the patient as a Provider.

With the more complete EHR data in the pull reply 156 from the Provider 122, the Aggregator 126 updates the basic EHR data obtained from the payment request trigger and stored in the basic EHR memory vault 94. The updated and more complete EHR data constitutes the augmented EHR record of the Healthcare delivered to the identified patient by the identified Provider. The augmented EHR record for the patient is thereafter stored in the augmented EHR vault 96 (also shown in FIG. 2).

The previously described series of transactions and interactions is repeated each time a patient obtains Healthcare delivered by a Provider. Each new instance of a Provider delivering Healthcare results in updating the augmented EHR record of each patient, after the Provider submits the payment request 138 and the Payer transmits the payment request trigger 142 to the Aggregator 126. In this manner, the EHR record of each patient is automatically updated for each instance of an additional patient-Provider transaction 128. The augmentation of the patient's EHR record based on the Healthcare previously delivered to the patient establishes a historically more-complete and contemporaneous augmented EHR record.

The augmented EHR record of each patient is stored in the augmented EHR vault 96 and is accessible to a Provider 122 for use in conjunction with delivering future Healthcare. When a new or existing patient 120 requests Healthcare from a new or existing Provider 122, a patient-Provider transaction 128 is initiated. The Provider 122 authenticates the patient by obtaining the patient's identification. Then, as part of the patient-Provider transaction 128, the Provider 122 sends an EHR request 168 to the Aggregator 126. The EHR request 168 includes the identification of the patient 120 and the identification of the Provider 122. The Aggregator 126 responds to the EHR request 168 by obtaining a copy of the augmented EHR record for the identified patient from the augmented EHR vault 96. An EHR reply 172 is communicated from the Aggregator 126 to the Provider 122 identified in the EHR request 168. The EHR reply 172 includes a copy of the augmented EHR record for the identified patient as exists in the augmented EHR vault 96.

Upon receiving the augmented EHR record for the identified patient in the EHR reply 172, the Provider 122 creates a local record of the augmented EHR and stores that record in the local memory 134 for that patient, if the patient is a new patient. If the patient is an existing patient, the Provider 122 updates the pre-existing local EHR record stored in the local memory 134 for that patient with the most current augmented EHR record contained in the EHR reply 172.

After updating the local EHR record of the patient with the augmented EHR record received from the Aggregator 126, and after delivering Healthcare to the patient, the Provider 122 again updates the local EHR record to reflect the Healthcare delivered. That updated EHR record is then stored in local memory 134. When the

Provider 122 sends a payment request 138 to a Payer 124 to receive compensation for the Healthcare delivered, the previously described actions which lead to augmenting the patient's EHR data commence, so that the updated local EHR record in the local memory 134 is transmitted to the Aggregator 126 as part of a pull reply 156 to the EHR Aggregator 126. The most current information from the EHR data received in the pull reply 156 is used by the Aggregator 126 to augment the EHR record of the patient stored in the augmented EHR vault 96. In this manner, a contemporaneous, comprehensive and augmented EHR record for the patient is established in the augmented EHR vault 96 for each patient and that augmented EHR record becomes available to use in executing the procedure 20 (FIGS. 1A-1D).

The MU-compliant information describing the augmented EHR of the patient is readily available to the Aggregator 126. No initiatives from the patient or further efforts from the Provider are required to collect and augment the EHR data of the patient. The payment requests 138 and the payment request triggers 142 constitute a reliable basis for collecting, aggregating and augmenting EHR data of the patient stored in the augmented EHR vault 96.

For purposes of clarity of description, each payment request 138, each payment request trigger 142, each pull request 152, a each pull reply 156, each EHR request 168 and each EHR reply 172 is shown as a separate and direct communication between the entities involved. In actual practice, these communications are performed over a public communication network, such as the internet 106 (FIG. 2). Such communications are possible because of the unique public network addresses of the Providers 122, the Payers 124, and the Aggregator 126. These communications, although shown as direct, can occur through intermediate entities such as clearing houses and health information exchanges.

The Provider status of the Aggregator 126 may be obtained with the consent of the patient 120 as part of the patient-Provider transaction 128, for example. Provider status of the Aggregator under the MU standards may also be negotiated with governmental regulatory bodies. To become a Provider, the Aggregator must provide Healthcare under the MU standards, such as, for example, establishing and providing diagnostic and health monitoring services to the patient in his or her home. The benefit of having the augmented EHR data available for use by Providers is an incentive for patients to authorize the Aggregator as a Provider in the patient-Provider transaction 128. The Aggregator 126 may also directly obtain authorization as a Provider from the patient. To obtain the status of a Provider, the Aggregator must obtain the certifications applicable to Providers.

In summary, the Basic EHR vault 94 is populated, without patient consent, by the Aggregator 126 procuring Healthcare information from Payers. Next, for those patients who have affirmatively agreed to use the Aggregator 126 as a Provider, the Aggregator 126 procures details of the EHR and procedures of the patient from other Providers, using the payment request as a trigger, and thereby populates the Augmented EHR Vault 96 with the procured information. Once the patient engages in this manner with the Aggregator 126, some of the patients will also provide additional pathologies or information to populate the genomic vault 98 and other vaults 99.

Communication between the Providers 122, the Payers 124 and the Aggregator 126 is facilitated by the Health Insurance Portability and Accountability Act of 1996 (HIPAA). HIPPA establishes a standard for Electronic Data Interchange (EDI) between Providers and Payers. The EDI Healthcare Claims Transaction Set (HIPAA Transaction 837) establishes a prevalent and widely utilized template for the components of the payment requests 138 from a Provider to a Payer.

The Aggregator 126 can use the ANSI X12N 270/271 Healthcare Eligibility Benefit Inquiry and Response transaction regulations to obtain additional information about the patient. The Aggregator sends an ANSI X12N 270 request to the Payer with the Payer health identification number (PHIN), patient's name and patient's date of birth. The Payer responds in a HIPAA 271 communication, which provides the Aggregator with the patient's address including city, state and zip code as well as the patient's gender. The Aggregator may use this enhanced patient identification information as part of its database to ensure accurate collection of the EHR data for the patient.

At the present time in United States, there are six Payers who offer Healthcare insurance or Healthcare payment coverage to approximately 170 million patients. Those Payers are CMS (Medicare), United Healthcare, Wellpoint, Aetna, Cigna and

Humana. Consequently at the present time, the Aggregator 126 needs only to acquire familiarity with a few different formats of payment requests 138 to extract information from the payment request triggers 142 which is beyond the purview of the HIPPA standards.

For pharmacy, dental, medical laboratory and other healthcare entities, there are specific transaction definitions similar to the HIPAA 837. For example in a retail pharmacy claim transaction, a National Council for Prescription Drug Programs (NCPDP) telecommunication standard is used as the basis for EDI payment requests 138 from pharmacy Providers to Payers. The details of these EDI transaction protocols vary but the basic information communicated is similar, and always includes a patient identification, a Provider identification, and a basic description of the Healthcare delivered.

Aspects of the basic EHR data which the Aggregator may employ in pull requests, and which are also contained in payment requests 138 and repeated in payment request triggers 142, include an International Classification of Diseases (ICD) code which indicates the disease or condition treated by the Provider, a Current Procedural Terminology (CPT) code which describes the medical procedure performed by the Provider on the patient, a National Drug Code (NDC) which describes the drug prescribed, the dosage of the drug, and the date when the Healthcare was delivered to the patient. The ICD, CPT and NDC codes and dates are a consistent set of definitions utilized by Payers and Providers.

It is advantageous for the historically complete EHR record of the patient to be available in the augmented EHR vault 96. Augmenting EHR record of a patient in response to a payment request ensures that the patient's EHR record is updated whenever a Provider delivers Healthcare to the patient. There are number of techniques for obtaining historical EHR data to include in the augmented EHR record.

Payment requests 138 submitted to Payers 124 are typically maintained by Payers for a considerable length of time, for example fourteen years. Consequently, the PHIN for a patient can be used by the Aggregator to access the historical basic EHR records of the patient stored by the Payer in connection with previous payment requests. Those historical records can thereafter be used to augment the EHR record, and to send pull requests 152 to the Providers that delivered the Healthcare. Provided that the historical local EHR records of the Providers are MU-compliant, those records are collected and incorporated in the patient's augmented EHR record.

Patients can also submit to the Aggregator 126 other records which contain information that describes historical Healthcare delivered. The Aggregator 126 will augment the patient's EHR record based on those records. For example, Healthcare delivery information is available from a so-called “super bill” that is generated by a Provider at the time of delivering Healthcare. The super bill includes much detail concerning the Healthcare delivered to the patient, and frequently includes codes which are MU-compliant. A Provider may give a copy of the super bill to the patient, and the patient can then supply the super bill to the Aggregator 126. The medical information from the super bill is then aggregated into the augmented EHR record by the Aggregator.

In those limited circumstances where patients have established and maintain a personal health record (PHR), the patient may give the Aggregator 126 access to the PHR. The medical information contained in the PHR is then used by the Aggregator to augment the EHR records of the patient stored in the augmented EHR vault 96.

Only a few laboratory medical service entities and pharmacies cover most of the laboratory and pharmacy services offered to patients in the United States. For laboratory medical services in the United States, Quest Diagnostics and Labcorp presently have the substantial majority market share of non-hospital based laboratory testing. The Aggregator 126 can periodically send pull requests 152 to these two companies for laboratory testing EHR data pertaining to a patient. The information contained in any pull reply 156 typically identifies those Providers which ordered the medical tests, and the Aggregator can further send pull requests 152 to those identified Providers. In the case of pharmacy services, a United States entity known as Surescripts acts as a clearinghouse for electronic prescriptions from a Provider to a retail pharmacy. The significant majority of prescriptions in the United States are funneled through Surescripts. The Aggregator can send pull requests 152 to Surescripts or to other similar intermediaries by which to obtain augmented EHR data. This information will again include the identities of prescribing Providers to which the Aggregator 126 can send further pull requests 152.

Typically and as described above, the Clinical Trial Entity will maintain its own computer system 91 (FIG. 2) and perform those functions of designing, enrolling, qualifying and conducting the clinical trial, while the Aggregator will maintain its own computer system 90 (FIG. 2) and perform those functions of aggregating the EHR data of the patients, matching patient etiologies with clinical trial requirements and soliciting matched patients to participate in the clinical trial, and possibly assisting the Clinical Trial Entity in enrolling patients in the clinical trial who respond favorably to solicitations. However, it is also possible that the functions and computer systems of the Clinical Trial Entity and the Aggregator could be combined and all of the functionality described in the present invention performed by a single entity.

The details of executing the procedure 20, shown in FIGS. 1A-1D, are now better described based on the information explained in connection with FIGS. 2 and 3.

The details of selecting specific clinical trial criteria, shown at 24 in FIG. 1A, involve the Administrator and medical experts employed by the Clinical Trial Entity selecting the specific diseases, human characteristics and health and medical conditions which define the patient for which the new medical therapy is intended to be applied. A patient with these characteristics and conditions who participates in the clinical trial will develop the best evidence of efficacy of the new medical therapy. The clinical trial criteria are selected from among the descriptions of the characteristics and conditions of patients in the medical records of the patients stored in the vaults 94, 96, 98 and 99.

The Administrator and medical experts employed by the Clinical Trial Entity, initially select the clinical trial criteria at 24. The Administrator and medical experts also approve changing the trial criteria when adjustments of the clinical trial occur at 34, 48, 54, 62, 68, 76 and 82. Changing the clinical trial criteria in this manner facilitates achieving meaningful outcomes when conducting the clinical trial.

The details of comparing patient medical records and the clinical trial criteria shown at 26, and determining the actual number of suitable patients with matching criteria shown at 28, involve typical computational activities executed by the Aggregator, such as set operations (union, intersection and difference) linked by patient identifications. Many of the procedural stages involved in designing the clinical trial by executing the procedure 20 will be accomplished without specifically identifying the patient. In such cases, for example when procuring patient counts and other associated information, a unique anonymized patient identification known only to the Aggregator may serve as the primary key for identifying the patients.

The details involved in extrapolating the actual number of suitable patients to represent the entire market, shown at 30 (FIG. 1A), is achieved by using statistically representative ratios of the number of patients whose medical records are contained in each of the three vaults 92, 94 and 96 (FIGS. 2 and 3). An example of developing simplified statistically representative number is explained as follows.

Assume that the specific trial criteria identifies N potential qualifying patients in the genome vault 96. These N patients directly correspond to N patients in the augmented EHR vault 94 and in the basic EHR vault 92, because the more specific medical records of the N patients in the genome vault would also fall within the less specific information contained in the basic EHR vault and in the augmented EHR vault. Specific clinical trial criteria would therefore have a statistically and empirically derived adoption ratio of AR[A] for the augmented EHR Vault and an adoption ratio of AR[B] for the basic EHR Vault. The adoption ratio AR[A] indicates that for these specific N patients and their profile, there would be AR[A] patients in the augmented EHR vault for each patient that exists in the genome vault, and there would be AR[B] patients in the basic EHR vault for each patient that exists in the augmented EHR vault.

Using these adoption ratios, starting with the N patients in the genome vault that specifically meet the trial criteria, the extrapolated number of patients (T) for market feasibility would be T={N×AR[A]}×AR[B]. N is the number of patients in the genomic vault with matching clinical trial criteria, and N×AR[A] represents the extrapolated number of patients in the augmented EHR vault with matching clinical trial criteria, and {N×AR[A]}×AR[B] represents the extrapolated number of patients in the basic EHR vault with matching clinical trial criteria.

N×AR[A] is greater than N because not all patients in the augmented EHR vault have had their genome sequenced and represented in the genome vault. Similarly, the number of patients in the basic EHR vault ({N×AR[A]}×AR[B]) is greater than the number of patients in the augmented EHR vault (N×AR[A]) because not all of the patients in the basic EHR vault have had their medical records augmented to the specificity required for inclusion in the augmented EHR vault. The value T represents the number of individuals within the general population which represent a commercial market for the newly developed therapy.

In this example, the adoption ratios AR[A] and AR[B] vary depending on disease states, age, gender, geography, income levels, etc. Pattern mapping algorithms can cluster variables to predictively refine the adoption ratios. Surveys can also be used to more accurately define the ratios.

The details of determining economic feasibility shown at 32 constitute the starting point to evaluate developing a new medical therapy, particularly a specific medical therapy which branches from a baseline therapy. The extrapolated number obtained at 30 is compared to the number of patients expected to consume the new therapy multiplied by the profit margin of marketing the new therapy. If profitability is demonstrated, the feasibility of researching and developing the new therapy is indicated by an affirmative determination at 32. The determining factor is the number of patients in the general population who are potential consumers of the new therapy, as derived at 30.

The determination at 32 involves the extrapolated number obtained at 30 and the expected profitability of the new therapy on a per patient basis compared to the anticipated cost of developing, testing and obtaining market approval for the use of the new therapy. If the comparison is inadequate to achieve economic feasibility, as indicated by the no (1) negative determination at 32, a decision is made at 34 to adjust the clinical trial criteria. If after iterations of adjusting the clinical trial criteria, economic feasibility is still not achieved, a no (2) negative determination is made at 32. That determination leads to the decision to wait at 38 and 40, or to terminate the entire procedure 20 at 42, as previously described. Of course, if the determination at 32 is affirmative, indicating favorable economic feasibility, the procedure 20 continues.

As a practical matter, once economic feasibility has been determined as indicated by the affirmative determination at 32, the subsequent determinations and evaluations within the procedure 20 involve the practicalities of assuring adequate participation to design, conduct and conclude the clinical trial successfully, while minimizing costs and achieving reliable data by which to evaluate efficacy. Nevertheless, the test for economic feasibility at 32 repeated in the procedure 20 with each dynamic adjustment of the clinical trial criteria when designing the clinical trial. In general, if economic feasibility is not demonstrated, it is unlikely that the clinical trial will progress to completion.

Next in the procedure 20, those suitable patients with matching characteristics and health and medical conditions, i.e. matching etiologies, that were determined at 26 and 28 are counted at 44 (FIG. 1B). The patients counted at 44 are those who are visible within the vaults 94, 96, 98 and 99 (FIG. 2). The patients counted at 44 constitute the maximum number that can be considered as suitable participants in the clinical trial under the present set of specific trial criteria selected at 24.

The number of suitable patients counted at 44 must exceed a minimum threshold in order to conduct a successful clinical trial, as determined by the Administrator and medical experts employed by the Clinical Trial Entity. The minimum threshold number of participants necessary to conduct a successful clinical trial is information which the Administrator and experts employed by the Clinical Trial Entity will determine in accordance with previously obtained heuristic experience in designing clinical trials, or in accordance with normally accepted standards for designing and successfully concluding clinical trials. The sufficiency of the counted number of suitable patients is determined at 46, by comparing the counted number of suitable patients at 44 with the minimum number of participants necessary to conduct the clinical trial. The threshold aspects of the determination at 46 center around the practical aspects of conducting the clinical trial to a successful completion.

If the number of suitable patients counted at 44 does not meet the minimum threshold for a successful clinical trial, as represented by a no (1) negative determination at 46, a decision is made by the Administrator and experts at 48 to adjust the clinical trial criteria selected at 24, in order to evaluate whether an adequate number of suitable patients exist to conduct a successful clinical trial. An affirmative determination at 48 represents the decision to adjust or change one or more of the specific clinical trial criteria selected at 24. The Administrator and experts of the

Clinical Trial Entity must approve the adjustment or change in the specific clinical trial criteria by selecting new specific clinical trial criteria at 24. Of course, no adjustments will be necessary if the number of suitable patients initially counted at 44 is adequate, as represented by initial affirmative and negative determinations at 46 and 48, respectively.

Once the clinical trial criteria has been changed or adjusted, the medical records of the patients are compared to the newly selected clinical trial criteria by the Aggregator at 26, and the number of suitable patients with matching criteria are determined at 28. Economic feasibility is determined by the Clinical Trial Entity at 30, 32 and 34. The number of suitable patients with etiologies matching the newly adjusted criteria are counted at 44 and the sufficiency of this number is again tested at 46 and 48.

Dynamic adjustment of the clinical trial criteria continues in this manner until the sufficiency threshold at 46 is met, as represented by an affirmative determination at 46. On the other hand, after a number of iterative attempts at adjusting the clinical trial criteria to achieve an adequate number of suitable patients proves impossible to accomplish, a no (2) negative determination at 46 leads to a choice by the Clinical Trial Entity of whether to wait at 38 for a predetermined amount of time to expire as determined at 40, or to end the procedure 20 at 42. The benefit of waiting is that new patients with medical records matching the adjusted clinical trial criteria might become available in the database, either because of the addition of new patients in the vaults 94, 96, 98 and 99 (FIG. 2), or because the characteristics and health conditions of enough existing patients in the vaults 94, 96, 98 and 99 (FIG. 2) has changed.

Although the dynamic adjustment at 46 and 48 described above is in terms of increasing the number of suitable patients, the same type of dynamic adjustment may be employed to reduce the number of suitable patients, if an excessive number of such patients are counted.

Upon meeting the threshold at 46 of attaining an appropriate number of suitable patients with matching etiologies, the procedure 20 moves to 50 and 52, where the number of suitable patients identified at 44 is evaluated by the Administrator and experts employed by the Clinical Trial Entity as constituting an adequate pool of potential participants to qualify for and successfully complete the clinical trial. The evaluation at 50 and the determination at 52 involve applying a reduction factor to the number of suitable patients counted at 44. The reduction factor takes into account that less than all of the suitable patients counted at 44 will qualify for and complete the clinical trial. For example, not all of the suitable patients will respond to a solicitation to participate, and of those who do respond favorably, not all will enroll as participants. Not all of those suitable patients who enroll as participants will qualify as participants under the very strict qualification laws and regulations applicable to clinical trials. Of those qualified and enrolled patients, a certain number will drop out of the clinical trial before it is completed. All of these reductions are taken into account in the reduction factor applied at 50. The reduction factor is an estimate, and is typically based on the empirical experience gained by the Clinical Trial Entity in designing and conducting clinical trials.

The purpose of the evaluation at 50 and the determination at 52 is to make a practical prediction of the participation, before any suitable patients are solicited for participation. By making the evaluation at 50 and the determination at 52, before soliciting the suitable patients, inefficiencies, delays and failures are avoided when designing the later stages of the clinical trial and conducting the clinical trial. The evaluation at 50 and the determination at 52 are primarily matters of practical efficiency in designing and conducting the clinical trial.

The determination at 52 is whether the number of qualified patients identified at 44 meets a threshold after the reduction factor has been applied. If the number is inadequate to achieve an adequately sized pool of qualified patients, or if the pool of qualified patients is excessive, as determined by a no (1) negative determination at 52, an affirmative decision at 54 results in adjusting the clinical trial criteria with the expectation of increasing or decreasing the pool of suitable patients, as the case may be. Increasing the pool of suitable patients facilitates successfully completing the clinical trial, while decreasing the pool of suitable patients reduces the cost of the clinical trial.

If after iterations of adjusting the clinical trial criteria in the manner described does not achieve an adequate pool of suitable patients, a no (2) negative determination at 32 will be made. That determination at 32 leads to the decision to wait at 38 and 40, or to end the procedure 20 at 42. Of course, if the dynamic adjustment of the clinical trial criteria results in an adequate pool of suitable patients, an affirmative determination at 52 results in a determination at 54. Thereafter the suitable patients are identified at 55, and the identified patients are solicited by the Liaison of the Aggregator to participate in the clinical trial at 56. The Liaison solicits the identified patients to protect patient privacy. If the solicited patient agrees to participate in the clinical trial and also agrees to the disclosure of his or her confidential and protected medical information to the Clinical Trial Entity, communications with each consenting solicited patient may be assumed by the Administrator of the Clinical Trial Entity. This solicitation of the patient by the Liaison 102 is preferably fully automated. A favorable response from the patient next triggers an automated process that links the patient with the Clinical Trial Entity.

Directly soliciting the identified patients at 56 is the first instance in the procedure 20 where patients have any notice or information concerning the possibility of their participation in a clinical trial. Designing the previous stages of the clinical trial in the manner described permits the Clinical Trial Entity to reform the clinical trial criteria without involving patients and without disclosure of the clinical trial. Dynamically adjusting the scope of the clinical trial to achieve the best efficiency without compromising the end results, and doing so while maintaining secrecy, are commercial benefits which the Clinical Trial Entity typically wishes to preserve.

As part of the patient's relationship with the Aggregator (126, FIG. 3), the patient provides an internet address or a physical address that is used by the Liaison of the Aggregator to communicate with the patient. This address is subsequently used at 56 to inform a patient of the applicability of a clinical trial and solicit his or her agreement to participate in the trial. The Clinical Trial Entity is not involved in this solicitation. The Aggregator, acting as a Provider to the patient, makes this solicitation. In this regard the solicitation complies with patient privacy laws and regulations. As an important consequence, this direct, non-intermediated, and typically automated solicitation occurs without compromising the identity or protected health information of the patient.

Directly soliciting the identified patients at 56 involves the Liaison of the Aggregator sending each identified patient an invitation to participate in the clinical trial. The solicitation is algorithmically generated and preferably sent electronically directly to the patient over the internet 106 (FIG. 2). The Liaison will issue solicitations to those identified patients who do not communicate over the internet using other forms of communication, such as regular mail. The efficiency of designing the clinical trial is greatly facilitated if the identified patients have the capability of communicating over the internet.

Directly soliciting participation of the patients at 56 is possible because the Aggregator is a Provider, and Providers may communicate directly with patients without intermediation. Eliminating intermediation by use of direct communication, while preserving patient privacy, increases the efficiency of designing the clinical trial.

Favorable responses to the solicitations issued at 56 are accumulated and counted at 58. If a solicited patient elects not to participate, that patient is not further solicited. If any solicited patient does not respond within a predetermined time interval, that patient is again solicited. After the expiration of another time interval that would indicate that the solicited patient is not interested in participating, no further attempt is made to solicit that patient.

Periodically the counted number of favorable responses at 58 is tested against a threshold number at 60 (FIG. 1C). The threshold number used at 60 will be established by the Administrator and experts of the Clinical Trial Entity, taking into account empirical experience, heuristics and/or normally accepted standards concerning the extent of participation required in successful clinical trials, at this stage of designing the clinical trial. If the number of favorably responding patients is inadequate to conduct the clinical trial, or if there are an excessive number of patients responding favorably to the solicitation, as determined by a no (1) negative determination at 60, an affirmative decision is made at 62 to adjust the clinical trial criteria. Adjusting the clinical trial criteria will result in identifying a different number of qualified patients at 44, followed by evaluating at 30 and 32 and 50 and 52 whether the number of newly identified patients is sufficient to constitute an adequate pool of potential participants.

Additional suitable patients identified from the dynamic adjustment of the clinical trial criteria are then sent invitations to participate at 56 by the Liaison. Those additional patients who respond favorably are counted at 58. The determination is thereafter made at 60 as to whether, with inclusion of the additional favorably responding patients, an adequate pool of favorably responding patients exists at this stage of the procedure 20 to conduct a clinical trial. If not, the dynamic adjustment continues with an affirmative determination at 62.

On the other hand, if it is desired to reduce the number of favorably responding patients, an affirmative determination at 62 results in adjusting the clinical trial criteria by increasing the specificity of those criteria. The adjustment will eliminate those ones of the previously favorably responding patients who do not meet the changed criteria of the increased specificity. Those who do not meet the increased specificity of the clinical trial criteria are counted at 58. After an affirmative determination at 60, notices are then sent to those favorably responding patients who do not meet the adjusted clinical trial criteria, informing them that their participation in the clinical trial will not be required. Notifying patients that their participation will no longer be required is subsumed within the solicitation at 56.

If after iterations of adjusting the clinical trial criteria in this manner, adequate enrollment still has not been achieved, a no (2) negative determination at 60 leads to the decision by the Administrator and experts to wait at 38 and 40, or to terminate the procedure 20 at 42. Of course, if the determinations at 60 and 62 are affirmative and negative, respectively, indicating that adequate enrollment has been achieved, the procedure 20 continues to 64.

At 64 and 66, an evaluation is made by the Clinical Trial Entity as to whether the number of favorably responding patients counted at 58 constitutes an adequate pool of potential participants in the clinical trial to result in the successful enrollment, qualification and completion of the clinical trial. The evaluation at 64 and the determination at 66 involves applying a reduction factor to the number of favorably responding patients counted at 58. The reduction factor is an estimate which takes into account that less than all of the favorably responding patients counted at 58 will actually enroll as participants, will qualify as participants under the very strict qualification laws and regulations applicable to clinical trials, and will complete the clinical trial. All of these reductions are taken into account in the reduction factor applied at 64 by the Clinical Trial Entity. The reduction factor is an estimate, and is typically based on the empirical experience gained from designing and conducting clinical trials.

The reduction factor applied at 64 may differ from the reduction factor applied at 50, even though both reduction factors involve similar considerations. The reduction factor applied at 64 may be refined based on the degree of response to the solicitations represented by the responses counted at 58. This information was not available at the time that the reduction factor was applied at 50. Also, experience in designing clinical trials may demonstrate that adjustments to the reduction factors are appropriate at different stages of designing the clinical trial, based on different degrees of seriousness or imminency, or other factors that come into play and achieve significance as the clinical trial design nears completion.

The purpose of the evaluation at 64 and the determination at 66 is to enable the Clinical Trial Entity to make a practical prediction of the number of favorably responding patients who will actually enroll as participants in the clinical trial, before any of the favorably responding patients are solicited to enroll as participants. By making the evaluation at 64 and the determination at 66 before attempting to enroll the favorably responding patients, delays and inefficiencies are avoided. Some of the favorably responding patients will have changed their mind about participation in the clinical trial between the time when they favorably responded to a solicitation to participate and when they are solicited to enroll. The pool of favorably responding patients should be of adequate size to allow some of the favorably responding patients to withdraw from participation. The evaluation at 64 and the determination at 66 are primarily matters of practical efficiency in designing the clinical trial at this stage of the procedure 20, and ensure that the subsequent design activities are efficiently conducted without delaying or compromising the clinical trial.

The determination at 66 is whether the number of favorably responding patients counted at 58 meets a threshold after the reduction factor has been applied by the Clinical Trial Entity at this stage of designing the clinical trial. If the number is inadequate to achieve an adequately sized pool of patients who are likely to enroll and qualify for the clinical trial, or if the pool of patients is excessive, as determined by a no (1) negative determination at 66, an affirmative decision at 68 results in dynamically adjusting the clinical trial criteria with the expectation of increasing or decreasing (as the case may be) the pool of favorably responding patients who are likely to enroll. Increasing or decreasing the pool of favorably responding patients who are likely to enroll is desirable at this stage of the procedure 20 to reduce the cost of the clinical trial while still obtaining reliable results.

If after iterations of adjusting the clinical trial criteria in this manner and an adequate pool of identified and qualified patients who are likely to enroll has still not been achieved, a no (2) negative determination at 66 leads to the decision to wait at 38 and 40 or to end the procedure 20 at 42. Of course, respectively affirmative and negative determinations at 66 and 68 indicate that the pool of favorably responding patients who are likely to enroll is predicted as adequate to qualify and complete the clinical trial successfully.

The adjustment achieved as a result of the actions 64, 66 and 68 reduces the cost of the clinical trial. There is administrative cost involved in attempting to enroll and qualify patients as participants. Enrolling and qualifying patients generally requires human if not face-to-face interaction to arrange for and agree on appropriate terms for compensation. Paperwork, including informational forms and consents, are typically required and must be obtained. Enrolling and qualifying no more patients than is necessary to achieve meaningful results from the clinical trial facilitates the efficiency and reduces the cost of conducting the clinical trial.

Enrolling patients as participants in the clinical trial at 70 involves sending an invitation to enroll to each favorably responding and qualified patient in the pool previously established at 58, 60, 62, 64, 66 and 68. The enrollment invitation is preferably sent electronically to those patients communicating over the internet 106 (FIG. 2). However, in those cases where some of the patients do not communicate over the internet, the invitations may be issued using other forms of communication, such as regular mail.

A very important part of enrollment involves detailed contact to complete the terms of the enrollment. In addition to reaching an agreement with the patient to participate in the clinical trial, enrollment involves managing and completing certain qualification requirements as shown at 71. The qualification requirements are specified by law and regulation. These qualification requirements go well beyond the etiological conditions of the patients described in the database at 22. Qualification requirements involve such things as family support for the participant, adequate transportation for the patient to and from examinations and appointments, access to doctors and pharmacies, conflicts of interest and many other factors. To assure that a patient attempting to enroll meets these qualification requirements, human contact actions are required by the Clinical Trial Entity. Usually these human interactions are performed by the Administrator. Only those patients who successfully qualify are actually enrolled at 70. The Aggregator can also automatically screen and enroll patients for the Clinical Trial Entity and thereby reduce the manual interactions at this stage of the enrollment process.

Enrolling patients at 70 may be the first instance where the identity of the Clinical Trial Entity is disclosed. Control over the disclosure of clinical trials has commercial value, and minimizing the number of patients involved by use of the dynamic adjustment aspect of the procedure 20 helps protect that control and commercial value.

The number of enrolled participants is accumulated and counted at 72. The number of enrolled participants counted at 72 is evaluated at 74 against a threshold number (FIG. 1D). The threshold number used at 74 will be established by the

Administrator and experts employed by the Clinical Trial Entity. If the number of enrolled patients counted at 72 is inadequate to complete the clinical trial, or if there are an excessive number of patients enrolled, as determined by a no (1) negative determination at 74, an affirmative decision at 76 adjusts the clinical trial criteria to achieve the desired level of enrollment. Under this circumstance, adjusting the clinical trial criteria will result in progressing through the procedure 20, in the manner previously described.

The additional patients identified for enrollment as a result of the dynamic adjustment are then sent invitations to enroll at 70. Those favorably responding patients are counted at 72. The determination is thereafter made at 74 by the Clinical

Trial Entity whether adequate participants have enrolled to conduct the clinical trial. If it is desired to reduce the number of enrolled patients, after adjusting the clinical trial criteria, notices are sent to any previous enrolled patients who have been eliminated as a result of the dynamic adjustment, informing them that their participation in the clinical trial is not be required. Notifying the previous enrolled patients that their participation will no longer be required is subsumed within the enrollment activity at 70. Of course, those previous enrolled patients whose participation is no longer required are subtracted from the count at 72.

If after iterations of adjusting the clinical trial criteria in this manner, and adequate enrollment still has not been achieved, a no (2) negative determination at 74 by the Clinical Trial Entity leads to the decision to wait at 38 and 40 or to the end procedure 20 at 42, as previously described. Of course, if the determination at 74 is affirmative, indicating that the adequate enrollment has been achieved, the procedure 20 moves to 78 and 80.

At 78 and 80, an evaluation is made by the Clinical Trial Entity of whether the number of enrolled participants counted at 72 constitutes an adequate pool of participants to successfully complete the clinical trial. Not all of the enrolled participants in the clinical trial will complete the clinical trial, due to such things as death, sickness, health condition changes, geographical movement, and lack of interest. The evaluation at 78 involves applying a reduction factor to the number of enrolled participants counted at 72. The reduction factor is an estimate, and is typically based on empirical experience in observing the number of enrolled and qualified participants who typically complete a clinical trial.

The determination at 80 is whether the number of participants enrolled counted at 72, as reduced by the reduction factor set by the Clinical Trial Entity, meets a sufficient threshold. If the number is inadequate to achieve an adequately sized pool of enrolled and qualified participants, or if the pool of enrolled and qualified participants is excessive, a no (1) negative determination at 80 results in an affirmative decision at 82 to adjust the clinical trial criteria with the expectation of increasing or decreasing the pool of enrolled and qualified participants. Decreasing the pool of enrolled participants is desirable to reduce the cost of the clinical trial under circumstances where efficacy can still be reliably determined. Increasing the pool of enrolled participants is desirable to assure that the clinical trial can be successfully completed.

The purpose of the evaluation at 78 and the determination at 80 is to enable the Clinical Trial Entity make a practical prediction of the number of qualified participants to complete the clinical trial, before the clinical trial is commenced at 84. By making the determinations at 78 and 80, before starting the clinical trial, delays in completing the clinical trial or a premature termination of the clinical trial is avoided because enough enrolled and qualified participants exist to overcome the various factors which may prevent some of the participants from completing the clinical trial.

If after iterations of adjusting the clinical trial criteria in this manner and an adequate pool of enrolled and qualified participants has still not been achieved, a no (2) negative determination at 66 leads to a decision by the Clinical Trial Entity to wait at 38 and 40, or to end the procedure 20 at 42. Of course, if the determinations at 80 and 82 are affirmative and negative, respectively, indicating that the pool of enrolled and qualified participants is adequate to successfully complete the clinical trial, the design stages of the clinical trial procedure 20 have been completed. The clinical trial is thereafter conducted at 84 by the entity which actually conducts the clinical trial. The entity conducting the clinical trial may or may not be the Clinical Trial Entity, since in some cases the Clinical Trial Entity sets up the right patients to commence the trial and then hands over the remainder of the clinical trial to be conducted by another entity.

The previous description of the procedure 20 include instances at 46, 50, 52, 60, 74, and 80 which involve determining and evaluating whether a number of patients is acceptable at each of the different stages for soliciting participation, enrolling participants and completing the clinical trial. The determinations at 46, 60 and 74 are made by the Clinical Trial Entity and involve a comparison of actual counted numbers relative to threshold numbers. The evaluations at 50, 64 and 78 involve predictions based on counted numbers, again made by the Clinical Trial Entity. Instead of separate determinations and evaluations, the actions at 46, 50 and 52, and at 60, 64 and 66, and at 74, 78 and 80, could each be combined into a single determination which both counts and evaluates or predicts the outcome, before making a dynamic adjustment of the clinical trial criteria.

Executing the complete procedure 20 results in designing a clinical trial under circumstances which achieve economic feasibility, efficiency and cost reduction. Economic feasibility accurately predicts whether the cost and expense of researching and developing a new medical therapy is justified. The efficiency and cost reduction arise from the ability to dynamically adjust the clinical trial criteria and thereby change the number of participants to an optimal number of not substantially more and not substantially less than the number of participants required to efficiently complete a clinical trial which demonstrates efficacy. Dynamic adjustment also reduces the cost of the clinical trial, and enhances or ensures the probability of completing a clinical trial which yields results that allow the efficacy of a newly developed medical therapy to be effectively and efficiently evaluated. This level of non-intermediated identification, solicitation and enrollment, which complies with patient privacy and confidentiality laws and regulations, is believed to have been previously unavailable to clinical trial entities.

In addition to dynamic adjustment, the invention permits directly pushing clinical trial information and solicitations to qualified patients in a specifically targeted manner while maintaining the privacy and confidentiality of the patient. A targeted push of this nature is a significant improvement when compared with current techniques of pulling in patients based on a broad notice of a trial and the expectation that patients will find their way to the clinical trial on their own initiative or through an intermediated solicitation by a Provider. The combined status of the Aggregator, both as an Aggregator of full etiologies of many millions of patients, for example, and as a Provider of Healthcare to the same number of patients, allows the Aggregator to access the etiology Vaults for matches and then, in a dis-intermediated manner, solicit matched and qualified patients for enrollment. The combined Aggregator/Provider status permits these actions, with access to full etiologies of patients, while maintaining patient privacy and compliance with requirements as required by law.

These benefits are particularly important when a baseline therapy is extended or altered to treat patients with more specific etiological characteristics. In such circumstances, the clinical trial must be conducted using very specific clinical trial criteria to address deep levels of specificity of etiologies. Market feasibility is more uncertain under these circumstances, and suitable participants for the clinical trial are considerably reduced in number and much more difficult to identify and recruit. Iteratively adjusting different levels of specificity in terms of market feasibility, ready electronic accessibility and rapid enrollment facilitates developing genomically specific therapies. The procedure 20 facilitates overcoming these practical hurdles at all the stages involved in designing the clinical trial, with efficiency not previously available in other techniques for designing clinical trials. This efficiency allows for the current annual number of fifty thousand clinical trials involving about ten million participants to scale up to millions of annual clinical trials engaging hundreds of millions of participants. This scale will be necessary as baseline and other therapies are customized to treat more specific etiological and genomic variations.

Another benefit of the procedure 20 is that it is also scalable in terms of the size of the clinical trial conducted. Clinical trials with large number of participants are processed as straightforwardly with the procedure 20 as smaller clinical trials with fewer participants, all of which is facilitated by the automated processing and communication capabilities of the procedure 20.

The benefits and improvements of the present invention create significant improvements in clinical trial design, resulting in part from the ability to aggregate and utilize the medical records of a massive number of patients on a continuous and timely updated basis. This ability is made possible as a result of recognizing that the medical records can be aggregated by an entity which achieves the status of a Provider and which updates those medical records in response to payment requests sent by Providers to Payers, in compliance with patient privacy and confidentiality laws and regulations, as discussed in more detail in U.S. patent application Ser. No. 13/839,539. Many other benefits and improvements will be apparent upon gaining a full appreciation of the present invention.

The detail of the above description constitutes a description of a preferred example of implementing the invention. The detail of the preceding description is not intended to limit the scope of the invention except to the extent explicitly incorporated in the following claims. The scope of the invention is defined by the following claims.

The invention claimed is:

1. A method of designing a clinical trial, comprising:
aggregating patient medical records of multiple patients in a database, the medical record of each patient in the database including information describing characteristics and health conditions of each patient;
establishing the characteristics and health conditions in the database for a first group of patients by collecting basic EHR data of each patient from a Payer who previously compensated a Provider for delivering Healthcare to each patient in the first group;
establishing the characteristics and health conditions in the database for a second group of patients by collecting EHR data from a Provider for each instance of the Provider delivering Healthcare to the patient in response to the Provider submitting a payment request to a Payer, and aggregating the collected EHR data for each patient of the second group with any basic EHR data for each patient of the second group to create augmented EHR data for each patient in the second group, the patients in the second group having a relatively higher degree of specificity of characteristics and health conditions than the patients in the first group;
selecting clinical trial criteria for participants in the clinical trial from among the characteristics and health conditions of patients in the first and second groups;
accessing the database to identify suitable patients as participants in the clinical trial who have characteristics and health conditions which match the selected clinical trial criteria; and
designing the clinical trial by reference to the identified qualified patients.

2. A method as defined in claim 1, further comprising:
selecting the clinical trial criteria to include at least one characteristic and health condition specific to patients in the second group;
determining the relative number of patients in each of the first and second groups;
identifying suitable patients from the second group as participants in the clinical trial who have characteristics and health conditions which match the clinical trial criteria; and
determining feasibility for marketing a newly developed therapy to be tested by a clinical trial of identified patients obtained from the second group, by multiplying the number of identified patients in the second group by a ratio of the number of patients in the first group relative to the number of patients in the second group.

3. A method as defined in claim 1, further comprising:
establishing the characteristics and health conditions in the database for a third group of patients by including genomic information for each patient of the third group with the augmented EHR data for each patient of the second group, the patients in the third group having a higher degree of specificity of characteristics and health conditions than the patients in the second group.

4. A method as defined in claim 3, further comprising:
selecting the clinical trial criteria to include at least one characteristic and health condition specific to patients in the third group;
determining the relative number of patients in each of the first, second and third groups;
identifying suitable patients from the third group as participants in the clinical trial who have characteristics and health conditions which match the clinical trial criteria; and
determining feasibility for marketing a newly developed therapy to be tested by a clinical trial of identified patients obtained from the second group, by multiplying the number of identified patients in the third group by a ratio of the number of patients in the second group relative to the number of patients in the third group and thereafter multiplying that result by a ratio of the number of patients in the first group relative to the number of patients in the second group.

5. A method as defined in claim 1, further comprising:
identifying suitable first patients in the database who have characteristics and health conditions which match the selected clinical trial criteria;
determining that the number of identified suitable first patients is inadequate to continue designing the clinical trial;
changing at least one of the characteristics or health conditions of the clinical trial criteria to create reformed clinical trial criteria;
identifying suitable second patients from the database who have characteristics and health conditions which match the reformed clinical trial criteria, the second patients differing in number from the first patients; and
designing the clinical trial by reference to the second patients.

6. A method as defined in claim 5, further comprising:
applying the aforesaid actions in at least one of a first stage of designing the clinical trial which includes soliciting suitable patients to participate in the clinical trial, and in a second stage of designing the clinical trial which includes enrolling favorably responding solicited patients as participants in the clinical trial, and in a third stage of designing the clinical trial which includes evaluating whether adequate enrolled patients will complete the clinical trial.

7. A method as defined in claim 5, further comprising:
applying the aforesaid actions in each of a first stage of designing the clinical trial which includes soliciting suitable patients to participate in the clinical trial, and in a second stage of designing the clinical trial which includes enrolling favorably responding solicited patients as participants in the clinical trial, and in a third stage of designing the clinical trial which includes evaluating whether adequate enrolled patients will complete the clinical trial.

8. A method as defined in claim 5, further comprising:
determining that the number of identified patients is inadequate to continue designing the clinical trial in at least one of a first stage of designing the clinical trial which includes soliciting suitable patients to participate in the clinical trial, and in a second stage of designing the clinical trial which includes enrolling favorably responding solicited patients as participants in the clinical trial, and in a third stage of designing the clinical trial which includes evaluating whether adequate enrolled patients will complete the clinical trial;
continuously updating the patient medical records in the database;
waiting for the patient medical records to update after determining that the number of patients is inadequate;
identifying patients from the database who have characteristics and health conditions which match the clinical trial criteria after the patient records have updated; and
designing the clinical trial by reference to the patients identified after the patient records have updated.

9. A method as defined in claim 1, further comprising:
identifying suitable first patients in the database who have characteristics and health conditions which match the selected clinical trial criteria;
determining that the number of identified suitable first patients is inadequate to continue designing the clinical trial;
changing at least one of the characteristics or health conditions of the clinical trial criteria to create first reformed clinical trial criteria;
identifying suitable second patients from the database who have characteristics and health conditions which match the first reformed clinical trial criteria, the second patients differing in number from the first patients;
determining that the number of identified suitable second patients is inadequate to continue designing the clinical trial;
again changing at least one of the characteristics or health conditions of the clinical trial criteria to create second reformed clinical trial criteria;
identifying suitable third patients from the database who have characteristics and health conditions which match the second reformed clinical trial criteria, the third patients differing in number from the first patients and the second patients; and
designing the clinical trial by reference to the third patients.

10. A method as defined in claim 9, further comprising:
applying the aforesaid actions in at least one of a first stage of designing the clinical trial which includes soliciting suitable patients to participate in the clinical trial, and in a second stage of designing the clinical trial which includes enrolling favorably responding solicited patients as participants in the clinical trial, and in a third stage of designing the clinical trial which includes evaluating whether adequate enrolled patients will complete the clinical trial.

11. A method as defined in claim 9, further comprising:
applying the aforesaid actions in each of a first stage of designing the clinical trial which includes soliciting suitable patients to participate in the clinical trial, and in a second stage of designing the clinical trial which includes enrolling favorably responding solicited patients as participants in the clinical trial, and in a third stage of designing the clinical trial which includes evaluating whether adequate enrolled patients will complete the clinical trial.

12. A method as defined in claim 9, further comprising:
determining that the number of identified patients is inadequate to continue designing the clinical trial in at least one of a first stage of designing the clinical trial which includes soliciting suitable patients to participate in the clinical trial, and in a second stage of designing the clinical trial which includes enrolling favorably responding solicited patients as participants in the clinical trial, and in a third stage of designing the clinical trial which includes evaluating whether adequate enrolled patients will complete the clinical trial;
continuously updating the patient medical records in the database;
waiting for the patient medical records to update after determining that the number of patients is inadequate;
identifying patients from the database who have characteristics and health conditions which match the clinical trial criteria after the patient records have updated; and
designing the clinical trial by reference to the patients identified after the patient records have updated.

13. A method as defined in claim 1, further comprising:
identifying suitable first patients in the database who have characteristics and health conditions which match the selected clinical trial criteria;
determining that the number of identified suitable first patients is inadequate to continue designing the clinical trial;
continuously updating the patient medical records in the database;
waiting for the patient medical records to update after determining that the number of first patients is inadequate;
identifying suitable second patients from the database who have characteristics and health conditions which match the clinical trial criteria after the patient records have updated; and
designing the clinical trial by reference to the suitable second patients identified after the patient records have updated.

14. A method as defined in claim 13, further comprising:
applying the aforesaid actions in at least one of a first stage of designing the clinical trial which includes soliciting suitable patients to participate in the clinical trial, and in a second stage of designing the clinical trial which includes enrolling favorably responding solicited patients as participants in the clinical trial, and in a third stage of designing the clinical trial which includes evaluating whether adequate enrolled patients will complete the clinical trial.

15. A method as defined in claim 13, further comprising:
applying the aforesaid actions in each of a first stage of designing the clinical trial which includes soliciting suitable patients to participate in the clinical trial, and in a second stage of designing the clinical trial which includes enrolling favorably responding solicited patients as participants in the clinical trial, and in a third stage of designing the clinical trial which includes evaluating whether adequate enrolled patients will complete the clinical trial.

16. A method as defined in claim 13, further comprising:
identifying suitable first patients in the database who have characteristics and health conditions which match the selected clinical trial criteria;
determining that the number of identified suitable first patients is inadequate to continue designing the clinical trial;
changing at least one of the characteristics or health conditions of the clinical trial criteria to create reformed clinical trial criteria;
identifying suitable second patients from the database who have characteristics and health conditions which match the reformed clinical trial criteria after the patient medical records have updated, the second patients differing in number from the first patients; and
designing the clinical trial by reference to the second patients.
